(19) United States
(12) Patent Application Publication
(10) Pub. No.: US 2022/0154254 A1
(43) Pub. Date: May 19, 2022

(54) Intestinal Biomarkers For Gut Health In Domesticated Birds

(71) Applicant: DUPONT NUTRITION BIOSCIENCES APS, COPENHAGEN K (DK)

(72) Inventors: Alexander BEKELE-YITBAREK, Wilmington, DE (US); Marion BERNARDEAU, Caen (FR); Venessa EECKHAUT, MERELBEKE (BE); Kirsty GIBBS, Cheshire (GB); Filip VAN IMMERSEEL, MERELBEKE (BE)

(21) Appl. No.: 17/600,371

(22) PCT Filed: Mar. 31, 2020
(86) PCT No.: PCT/US2020/025930

§ 371 Date: Sep. 30, 2021

Related U.S. Application Data
(60) Provisional application No. 62/827,725, filed on Apr. 1, 2019.

(51) Int. Cl. C12Q 1/689

(52) U.S. Cl. CPC C12Q 1/689; G01N 2030/027; C12Q 2600/118; C12Q 2600/16

(57) ABSTRACT

Provided herein, inter alia, are methods for measuring and assessing intestinal health in poultry. The disclosed microbial biomarkers and associated methods for identifying and quantifying the same are reliable, rapid and, in some embodiments, non-invasive, and can be used to provide information with respect to the gut health of poultry, such as chickens.

### CROSS-REFERENCE TO RELATED APPLICATIONS

This application claims priority to U.S. Provisional Patent Application No. 62/827,725, filed Apr. 1, 2019, the disclosure of which is incorporated by reference herein in its entirety.

### FIELD OF THE INVENTION

Provided herein, inter alia, are methods for measuring and assessing intestinal health in domesticated birds.

### BACKGROUND

In poultry species, the gastrointestinal tract and intestinal-associated microflora not only are involved in digestion and absorption, but also interact with the immune and central nervous system to modulate health. The inside of the intestinal tract is coated with a thin layer of sticky, viscous mucous, and embedded in this mucus layer, are millions and millions of bacteria and other microbes. When the intestinal bacteria are in balance (i.e., the good bacteria outnumber the bad bacteria), the gut is said to be healthy. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. In settings of “dysbiosis” or disrupted symbiosis, microbiota functions can be lost or deranged, resulting in increased susceptibility to pathogens, altered metabolic profiles, or induction of proinflammatory signals that can result in local or systemic inflammation or autoimmunity. Thus, the intestinal microbiota of poultry plays a significant role in the pathogenesis of many diseases and disorders, including a variety of pathogenic infections of the gut such as coccidiosis or necrotic enteritis.

Quantifiable and easy-to-measure biomarkers for diagnosing or predicting the intestinal health of poultry do not yet exist but would be of tremendous value as a tool to monitor and/or prognose clinical and subclinical intestinal entities that cause or are correlated with performance problems in flocks and to evaluate control methods for intestinal health, independent of whether the triggers are derived from host, nutritional or microbial factors. The subject matter disclosed herein addresses these needs and provides additional benefits as well.

### SUMMARY

Provided herein, inter alia, are methods for measuring and assessing intestinal health in poultry. The disclosed microbial biomarkers and associated methods for identifying and quantifying the same are reliable, rapid and, in some embodiments, non-invasive, and can provide information with respect to the gut health of poultry, such as chickens.

Accordingly, in some aspects, provided herein are methods for determining the intestinal health status of a domesticated bird comprising: quantifying populations of one or more microorganism(s) in a fecal and/or intestinal content sample from the bird selected from the group consisting of: a microorganism from the Clostridiales vadinBB60 group family of microorganisms and a microorganism from the Pepto streptococcaceae family of microorganisms, wherein a decreased population of said one or more microorganism(s) in said fecal or intestinal content sample, when compared to the level found in fecal or intestinal content samples of healthy control animals, is an indicator of poor intestinal health. In some embodiments, the method further comprises quantifying populations of one or more (such as any of 1, 2, 3, 4, 5, 6, 7, 8, or 9) microorganism(s) in a fecal and/or intestinal content sample from the bird selected from the group consisting of: a microorganism from the genus Brevibacterium, Brachybacterium, Ruminiclostridium, Candidatus Arthromitus, Ruminococcus with the optional exception of Ruminococcus torques, Streptococcus, Shuttleworthia, Lachnospiraceae NK4A136 group, and Ruminococcaceae UCG-005, wherein a decreased population of said one or more microorganism(s) in said fecal or intestinal content sample, when compared to the level found in fecal or intestinal content samples of healthy control animals, is an indicator of poor intestinal health. In some embodiments of any of the embodiments disclosed herein, the intestinal content sample is obtained from ileum, colon, or caecum. In some embodiments of any of the embodiments disclosed herein, the method further comprises quantifying populations of one or more (such as any of 1, 2, or 3) microorganism(s) in an intestinal content sample from the bird selected from: a microorganism from the genus Defluviitaleaceae UCG-011, a microorganism from the genus Lachnoclostridium, or a microorganism from the Ruminococcus torques group, (a) wherein a decreased population of said one or more microorganism(s) obtained from the caecum, when compared to the level found in caecum samples of healthy control animals, is an indicator of poor intestinal health; and/or (b) wherein an increased population of said one or more microorganism(s) obtained from the colon, when compared to the level found in colon samples of healthy control animals, is an indicator of poor intestinal health. In some embodiments of any of the embodiments disclosed herein, the method further comprises quantifying populations of one or more microorganism(s) in an intestinal content sample from the bird a microorganism from the genus Lactobacillus, (a) wherein an increased population of said one or more microorganism(s) obtained from the caecum, when compared to the level found in caecum samples of healthy control animals, is an indicator of poor intestinal health; and/or (b) wherein a decreased population of said one or more microorganism(s) obtained from the colon, when compared to the level found in colon samples of healthy control animals, is an indicator of poor intestinal health. In some embodiments of any of the embodiments disclosed herein, the method further comprises quantifying populations of one or more microorganism(s) in a fecal and/or intestinal content sample from the bird selected from (a) a microorganism from the phylum Tenericutes and/or Firmicutes; (b) one or more microorganism from the phylum Verrucomicrobia and/or Bacteroidetes; (c) one or more (such as any of 1, 2, 3, or 4 or more) microorganism from the class Mollicutes RF39, Erysipelotrichales, Clostridiales, and/or Micrococcales; (d) one or more (such as any of 1, 2, or 3, or more) microorganism from the class Coriobacteriales, Verrucomicrobiales, and/or Bacteroidales (e) one or more (such as any of 1, 2, 3, 4, 5, 6, 7, 8, 9, or more) microorganism from the family Streptococcaceae, Defluviitaleaceae, Christensenellaceae, Erysipelotrichaceae, Lachnospiraceae, Ruminococcaceae, Dermabacteraceae, Brevibacteriaceae, and/or Dietziaceae; (f) one or more (such as any of 1, 2, 3, or 4 or more) microorganism from the family Eggerthellaceae, Akkermansiaceae, Lactobacillaceae, and/or Clostridiaceae; (g) one or more (such as any of 1, 2, 3, 4, 5, 6, 7, 8, 9, 10, 11, 12, or 13, or more) microorganism from the genus Roseburia, Harryflintia, Ruminococcaceae UCG-009, Coprococcus, Ruminococcaceae UCG-010, Ruminococcus, Christensenellaceae R-7 group, Erysipelatoclostridium, Ruminococcaceae NK4A214 group, Negativibacillus, Oscillibacter, Butyricicoccus, and/or Eisenbergiella; and/or (h) a microorganism from the genus Eggerthella, and/or Akkermansia, (1) wherein a decreased population of said one or more microorganism(s) from (a), (c), (e), and/or (h) in said fecal or intestinal content sample, when compared to the level found in fecal or intestinal content samples of healthy control animals, is an indicator of poor intestinal health; and/or (2) wherein an increased population of said one or more microorganism(s) from (b), (d), (f), and/or (g) in said fecal or intestinal content sample, when compared to the level found in fecal or intestinal content samples of healthy control animals, is an indicator of poor intestinal health. In some embodiments, the intestinal content sample is obtained from ileum and/or caecum. In some embodiments of any of the embodiments disclosed herein, the method further comprises quantifying populations of one or more (such as any of 1, 2, 3, 4, 5, 6, 7, 8, 9, 10, 11, 12, 13, 14, 15, 16, 17, 18, 19, or 20) microorganism(s) in a fecal and/or intestinal content sample from the bird selected from (a) a microorganism from the order Rhodospirillales; (b) a microorganism from the genus Helicobacter, Staphylococcus, Jeotgalicoccus, Ruminococcus, Marvinbryantia, Ruminococcaceae UCG-013, Enterococcus, Corynebacterium, and/or Subdoligranulum; and/or (c) a microorganism from the genus Firmicutes, Anaerofilum, Intestinimonas, Fournierella, Barnesiella, Barnesiella, Bifidobacterium, Tyzzerella, Clostridium sensu stricto, and/or Escherichia-Shigella, (1) wherein a decreased population of said one or more microorganism(s) from (a) and/or (b) in said fecal or intestinal content sample, when compared to the level found in fecal or intestinal content samples of healthy control animals, is an indicator of poor intestinal health; and/or (2) wherein an increased population of said one or more microorganism(s) from (c) in said fecal or intestinal content sample, when compared to the level found in fecal or intestinal content samples of healthy control animals, is an indicator of poor intestinal health. In some embodiments, the intestinal content sample is obtained from colon and/or caecum. In some embodiments of any of the embodiments disclosed herein, intestinal health is determined by one or more of (a) measuring villus length in the duodenum of the birds; (b) measuring villus-to crypt ratio in the duodenum of the birds; (c) measuring T-lymphocyte infiltration in villi; and/or (d) scoring the macroscopic gut appearance of the birds. In some embodiments of any of the embodiments disclosed herein, the domesticated bird is selected from the group consisting of chickens, turkeys, ducks, geese, emus, ostriches, quail, and pheasant. In some embodiments, the chicken is a broiler. In some embodiments of any of the embodiments disclosed herein, said one or more microorganism(s) are quantified by using antibodies which specifically bind to said microorganism. In some embodiments, said antibodies are part of an Enzyme-Linked Immuno Sorbent Assay (ELISA). In some embodiments of any of the embodiments disclosed herein, said one or more microorganisms are identified and quantified by real-time PCR. In some embodiments, the method further comprises sequencing the 16S ribosomal DNA (rDNA) gene. In some embodiments of any of the embodiments disclosed herein, the method further comprises quantifying one or more (such as any of 1, 2, 3, 4, 5, 6, 7, 8, 9, 10, 11, 12, 13, 14, 15, 16, 17, 18, 19, or 20) metabolite(s) in a fecal and/or intestinal content sample from the bird selected from the group consisting of linoleyl carnitine, linalool, 3-[(9Z)-9-octadecenoyloxy]-4-(trimethylammonio)butanoate, (−)-trans-methyl dihydrojasmonate, icomucret, 1,3-dioctanoylglycerol, ethyl 2-nonynoate, 4-aminobutyrate, 2-amino-isobutyrate, D-alpha-aminobutyrate, cadaverine, putrescine, uracil, hypoxanthine, D-alanine, sarcosine, methional, hexanal, malondialdehyde, L-alanine, and acetylcarnitine, wherein an increased level of said one or more metabolite(s) in said fecal or intestinal content sample, when compared to the level found in fecal or intestinal content samples of healthy control animals, is an indicator of poor intestinal health. In some embodiments of any of the embodiments disclosed herein, the method further comprises quantifying one or more (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45) metabolite(s) in a fecal and/or intestinal content sample from the bird selected from the group consisting of 5-(2-carboxyethyl)-2-hydroxyphenyl beta-D-glucopyranosiduronic acid, 4,15-Diacetoxy-3-hydroxy-12,13-epoxytrichothec-9-en-8-yl3-hydroxy-3-methylbutanoate, scoparone, asp-leu, ethyl benzoylacetate, L-(+)-glutamine, 1-allyl-2,3,4,5-tetramethoxybenzene, (DL)-3-O-methyldopa, dictyoquinazol A, 1-(3-furyl)-7-hydroxy-4,8-dimethyl-1,6-nonanedione methyl 3,4,5-trimethoxycinnamate, butylparaben, aspartic acid, L-arginine, glutamic acid, L-pyroglutamic acid, L-glutamine, L-histidine, glycine, (−)-beta-pineen, L-asparagine, L-homoserine, L-serine, L-threonine, L-prdine, L-tyrosine, L-leucine, dopamine, taurocholic acid, tryptamine, tauroursodeoxychdic acid, glycoursodeoxycholic acid, ursodeoxycholic acid, cholic acid, nonanal, 3-methyl-2-butenal, DL-glyceraldehyde, allantoin, nicotinic acid, N-acetylglucosamine, spermidine, (dimethylamino)acetonitrile, glycoursodeoxycholic acid, tauroursodeoxycholic acid, cortisol, and heptanal, wherein a decreased level of said one or more metabolite(s) in said fecal or intestinal content sample, when compared to the level found in fecal or intestinal content samples of healthy control animals, is an indicator of poor intestinal health. In some embodiments of any of the embodiments disclosed herein, said one or more metabolite(s) are quantified by using antibodies which specifically bind to said metabolite. In some embodiments, said antibodies are part of an Enzyme-Linked Immuno Sorbent Assay (ELISA). In some embodiments of any of the embodiments disclosed herein, said one or more metabolite(s) are quantified by using mass spec or HPLC.

In other aspects, provided herein is a method for quantifying one or more microorganism(s) from a domesticated bird at risk for or thought to be at risk for poor intestinal health comprising: quantifying one or more microorganism(s) in a sample selected from the group consisting of a microorganism from the Clostridiales vadinBB60 group family of microorganisms and a microorganism from the Peptostreptococcaceae family of microorganisms, wherein the sample is a fecal or an intestinal content sample. In some embodiments, the method further comprises quantifying populations of one or more (such as any of 1, 2, 3, 4, 5, 6, 7, 8, or 9) microorganism(s) in the sample from the bird selected from the group consisting of: Brevibacterium, Brachybacterium, Ruminiclostridium, Candidatus Arthromitus, Ruminococcus with the optional exception of Ruminococcus torques, Streptococcus, Shuttleworthia, Lachnospiraceae NK4A136 group, and Ruminococcaceae UCG-005. In some embodiments of any of the embodiments disclosed herein, the intestinal content sample is obtained from ileum, colon, or caecum. In some embodiments of any of the embodiments disclosed herein, the method further comprises quantifying populations of one or more (such as any of 1, 2, or 3) microorganism(s) in an intestinal content sample from the bird selected from: a microorganism from the genus Defluviitaleaceae UCG-011, a microorganism from the genus Lachnoclostridium, a microorganism from the genus Lactobacillus, or a microorganism from the Ruminococcus torques group, wherein the intestinal content sample is obtained from colon or caecum. In some embodiments of any of the embodiments disclosed herein, the method further comprises quantifying populations of one or more microorganism(s) in a fecal and/or intestinal content sample from the bird selected from (a) one or more (such as any of 1, 2, 3, or 4 or more) microorganism from the phylum Tenericutes, Verrucomicrobia, Bacteroidetes, and/or Firmicutes; (b) one or more (such as any of 1, 2, 3, 4, 5, 6, or 7 or more) microorganism from the class Mollicutes RF39, Erysipelotrichales, Clostridiales, Coriobacteriales, Verrucomicrobiales, Bacteroidales, and/or Micrococcales; (c) one or more microorganism from the order Rhodospirillales; (d) one or more (such as any of 1, 2, 3, 4, 5, 6, 7, 8, 9, 10, 11, 12, or 13 or more) microorganism from the family Streptococcaceae, Defluviitaleaceae, Christensenellaceae, Erysipelotrichaceae, Lachnospiraceae, Ruminococcaceae, Dermabacteraceae, Brevibacteriaceae, Dietziaceae, Eggerthellaceae, Akkermansiaceae, Lactobacillaceae, and/or Clostridiaceae; and/or (e) one or more (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 or more) microorganism from the genus Roseburia, Harryflintia, Ruminococcaceae UCG-009, Coprococcus, Ruminococcaceae UCG-010, Ruminococcus, Christensenellaceae R-7 group, Erysipelatoclostridium, Ruminococcaceae NK4A214 group, Negativibacillus, Oscillibacter, Butyricicoccus, Eggerthella, Akkermansia, Helicobacter, Staphylococcus, Jeotgalicoccus, Ruminococcus, Marvinbryantia, Ruminococcaceae UCG-013, Enterococcus, Corynebacterium, Subdoligranulum, Firmicutes, Anaerofilum, Intestinimonas, Fournierella, Barnesiella, Barnesiella, Bifidobacterium, Tyzzerella, Clostridium sensu stricto, Escherichia-Shigella, and/or Eisenbergiella; wherein the intestinal content sample is obtained from colon and/or caecum. In some embodiments of any of the embodiments disclosed herein, the domesticated bird is selected from the group consisting of chickens, turkeys, ducks, geese, quail, and pheasant. In some embodiments, the chicken is a broiler. In some embodiments of any of the embodiments disclosed herein, said one or more microorganism(s) are quantified by using antibodies which specifically bind to said microorganism. In some embodiments, said antibodies are part of an Enzyme-Linked Immuno Sorbent Assay (ELISA). In some embodiments of any of the embodiments disclosed herein, said one or more microorganisms are identified and quantified by real-time PCR. In some embodiments, the method further comprises sequencing the 16S ribosomal DNA (rDNA) gene. In some embodiments of any of the embodiments disclosed herein, the method further comprises (a) measuring villus length in the duodenum of the birds; (b) measuring villus-to crypt ratio in the duodenum of the birds; (c) measuring T-lymphocyte infiltration in villi; and/or (d) scoring the macroscopic gut appearance of the birds. In some embodiments of any of the embodiments disclosed herein, the method further comprises quantifying one or more (such as any of 1, 2, 3, 4, 5, 6, 7, 8, 9, 10, 11, 12, 13, 14, 15, 16, 17, 18, 19, or 20) metabolite(s) in a fecal and/or intestinal content sample from the bird selected from the group consisting of linoleyl carnitine, linalool, 3-[(9Z)-9-octadecenoyloxy]-4-(trimethylammonio)butanoate, (−)-trans-methyl dihydrojasmonate, icomucret, 1,3-dioctanoylglycerol, ethyl 2-nonynoate, 4-aminobutyrate, 2-amino-isobutyrate, D-alpha-aminobutyrate, cadaverine, putrescine, uracil, hypoxanthine, D-alanine, sarcosine, methional, hexanal, malondialdehyde L-alanine, and acetylcarnitine, wherein the sample is a fecal or an intestinal content sample. In some embodiments of any of the embodiments disclosed herein, the method further comprises quantifying one or more (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45) metabolite(s) in a fecal and/or intestinal content sample from the bird selected from the group consisting of 5-(2-carboxyethyl)-2-hydroxyphenyl beta-D-glucopyranosiduronic acid, 4,15-Diacetoxy-3-hydroxy-12,13-epoxytrichothec-9-en-8-yl 3-hydroxy-3-methylbutanoate, scoparone, asp-leu, ethyl benzoylacetate, L-(+)-glutamine, 1-allyl-2,3,4,5-tetramethoxybenzene, (DL)-3-O-methyldopa, dictyoquinazol A, 1-(3-furyl)-7-hydroxy-4,8-dimethyl-1,6-nonanedione methyl 3,4,5-trimethoxycinnamate, butylparaben, aspartic acid, L-arginine, glutamic acid, L-pyroglutamic acid, L-glutamine, L-histidine, glycine, (−)-beta-pineen, L-asparagine, L-homoserine, L-serine, L-threonine, L-prdine, L-tyrosine, L-leucine, dopamine, taurocholic acid, tryptamine, tauroursodeoxychdic acid, glycoursodeoxycholic acid, ursodeoxycholic acid, cholic acid, nonanal, 3-methyl-2-butenal, DL-glyceraldehyde, allantoin, nicotinic acid, N-acetylglucosamine, spermidine, (dimethylamino)acetonitrile, glycoursodeoxycholic acid, tauroursodeoxycholic acid, cortisol, and heptanal. In some embodiments of any of the embodiments disclosed herein, said one or more metabolite(s) are quantified by using antibodies which specifically bind to said metabolite. In some embodiments, said antibodies are part of an Enzyme-Linked Immuno Sorbent Assay (ELISA). In some embodiments, said one or more metabolite(s) are quantified by using mass spec or HPLC.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1A is a bar graph depicting body weight (g) in control (ctrl.) and challenged chickens at day 28. FIG. 1B is a bar graph depicting coccidiosis and dysbiosis scores in control (ctrl.) and challenged chickens at day 28.

FIG. 2A is a plot depicting intestinal villus height (μm) in control (CTRL) compared to challenged chickens. FIG. 2B is a plot depicting crypt depth (μm) in control (CTRL) compared to challenged chickens. FIG. 2C is a plot depicting the ratio of villus height/crypt depth in control (CTRL) compared to challenged chickens.

FIG. 3A is a graph depicting the association between intestinal villus length (μm) and body weight (g) in challenged (dark) and control (light) birds. FIG. 3B is a graph depicting the association between intestinal crypt depth (μm) and body weight (g) in challenged (dark) and control (light) birds. FIG. 3C is a graph depicting the association between the ratio of villus height/crypt depth and body weight (g) in challenged (dark) and control (light) birds.

FIG. 4A is a plot depicting the area percentage of immune cell (CD3+) infiltration of intestinal tissue in control (CTRL) compared to challenged chickens. FIG. 4B is a graph depicting the association between the area percentage of immune cell (CD3, area %) infiltration of intestinal tissue with body weight (g) in challenged (dark) and control (light) birds. FIG. 4C is a graph depicting the association between the area percentage of immune cell (CD3, area %) infiltration of intestinal tissue with coccidiosis score in challenged (dark) and control (light) birds. FIG. 4D is a graph depicting the association between the area percentage of immune cell (CD3, area %) infiltration of intestinal tissue with dysbiosis score in challenged (dark) and control (light) birds. FIG. 4E is a graph depicting the association between the area percentage of immune cell (CD3, area %) infiltration of intestinal tissue with villus length (μm) in challenged (dark) and control (light) birds.

FIG. 5A depicts a graph showing a non-limiting example of a bacterium having a relative intestinal abundance that differs between challenged (dark) and control (light) birds as well as the association of relative abundance with villus length (μm). FIG. 5B depicts a graph showing a non-limiting example of the association between the relative abundance of two bacteria and the ratio of villus height/crypt depth. FIG. 5C depicts a graph showing a non-limiting example of the association between the relative abundance of three bacteria and the ratio of villus height/crypt depth. FIG. 5D depicts a graph showing a non-limiting example of the association between the relative abundance of a bacterium and the area percentage of immune cell (CD3, area percentage) infiltration of intestinal tissue.

FIG. 6A is a bar graph depicting body weight (g) in control (ctrl.) and challenged chickens at day 28. FIG. 6B is a bar graph depicting coccidiosis and dysbiosis scores in control (ctrl.) and challenged chickens at day 28.

FIG. 7A and FIG. 7B are bar graphs depicting the identity and quantity of non-limiting examples of metabolites measured in the colon (FIG. 7A) and caecum (FIG. 7B) of challenged and control birds.

FIG. 8A and FIG. 8B are bar graphs depicting the identity and quantity of non-limiting examples of metabolites measured in the colon (FIG. 8A) and caecum (FIG. 8B) of challenged and control birds.

FIG. 9 is a plot depicting the correlation between bacterial population of Ruminococcus torques group in the ceacum and body weight.

FIG. 10A, FIG. 10B and FIG. 10C are plots depicting the correlation between bacterial populations in the ceacum and CD3 area percentage.

FIG. 11A and FIG. 11B are plots depicting the correlation between bacterial populations in the ceacum and CD3 area percentage.

FIG. 12A and FIG. 12B are plots depicting the correlation between bacterial populations in the ceacum and CD3 area percentage.

FIG. 13A and FIG. 13B are plots depicting the correlation between bacterial populations in the ceacum and CD3 area percentage.

FIG. 14A and FIG. 14B are plots depicting the correlation between bacterial populations in the colon and CD3 area percentage.

FIG. 15A and FIG. 15B are plots depicting the correlation between bacterial populations in the colon and CD3 area percentage.

FIGS. 16A, FIG. 16B, and 16C are plots depicting the correlation between bacterial populations in the colon and CD3 area percentage.

FIG. 17A, FIG. 17B, FIG. 17C, FIG. 17D, FIG. 17E, FIG. 17F, FIG. 17G, FIG. 17H, FIG. 17I, FIG. 17J, and FIG. 17B, are plots depicting the correlation between bacterial populations in the colon and the ratio between villus length and crypt depth.

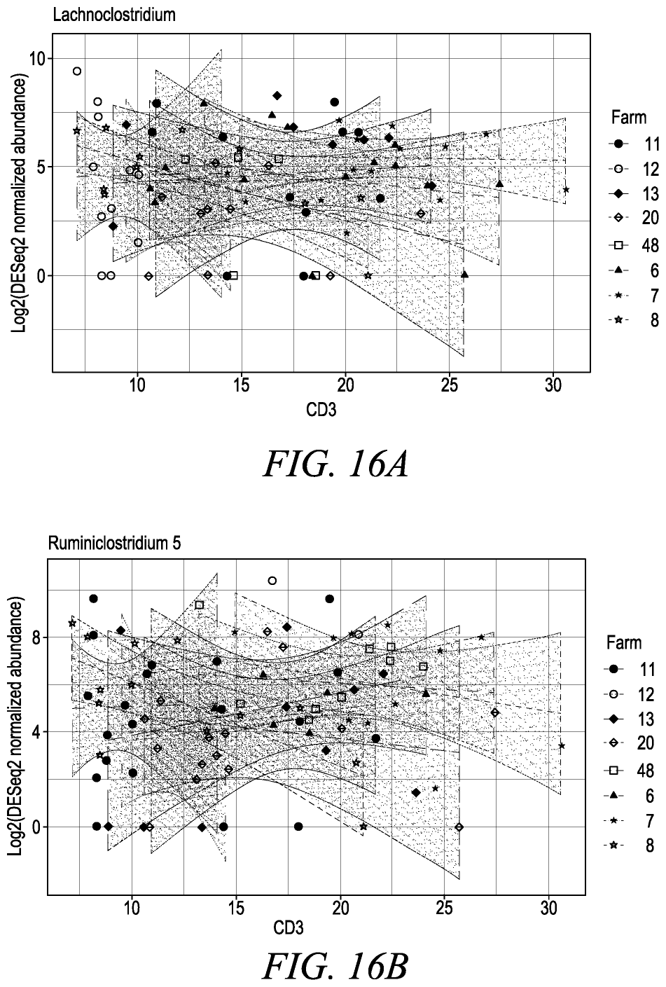

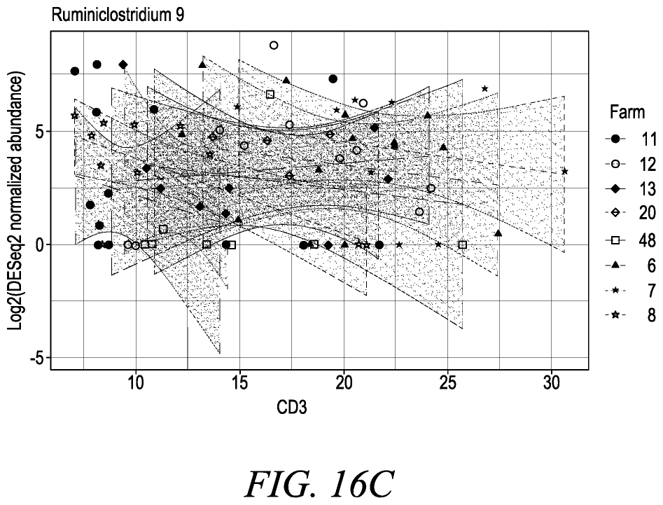

FIG. 16A, FIG. 16B, and 16C are plots depicting the correlation between bacterial populations in the colon and the ratio between villus length and crypt depth.

### DETAILED DESCRIPTION

For domesticated birds, particularly for birds bred for food production, a well-functioning intestinal tract is of key importance for digestion and nutrient absorption and consequently low feed conversion and is also crucial for health and welfare. Indeed, intestinal diseases and syndromes are common in some commercial forms of poultry, such as broilers, and constitute the most important cause for treatment (Casewell et al., 2003). In poultry fanning, coccidiosis is by far the most important intestinal disease (Yegani and Korver, 2008; Caly et al., 2015). Clinical diseases caused by bacterial pathogens are not common, but it is widely recognized that a variety of intestinal syndromes can affect broiler performance, including subclinical necrotic enteritis and coccidiosis, viral enteritis, and various non-defined enteritis syndromes (Yegani and Korver, 2008). It is not evident how to diagnose these subclinical entities and differentiate these from performance problems that have no infectious etiology, such as those caused by suboptimal formulated diets that not always cause intestinal damage.

The invention disclosed herein is based, inter alia, on the inventors' observations that the identity and quantity of constituent microorganisms in the gut (i.e., intestines) and feces of poultry varies in accordance with intestinal health status. As such, identifying and quantifying microbial species present in the chicken gut and/or fecal material can be used to monitor and/or prognose clinical and subclinical intestinal entities that cause or are correlated with performance problems (such as, but not limited to, decreased weight, poor feed conversion ratio (FCR), mortality, and altered intestinal structure and morphology).

I. Definitions

As used herein, “microorganism” refers to a bacterium, a fungus, a virus, a protozoan, and other microbes or microscopic organisms.

The phrase “increased population of a microorganism when compared to the level found in samples from healthy control animals” means at least a 10-200% increase, such as any of about a 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% increase, inclusive of all values falling in between these percentages. In some embodiments, the microorganism is not detectable at all in healthy control animals.

The phrase “decreased population of a microorganism when compared to the level found in samples from healthy control animals” means at least a 10-100% decrease, such as any of about a 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, or 100%, decrease, inclusive of all values falling in between these percentages. In some embodiments, the microorganism is not detectable at all in animals suffering from or thought to be suffering from poor intestinal health.

The term “poultry,” as used herein, means domesticated birds kept by humans for their eggs, their meat or their feathers. These birds are most typically members of the superorder Galloanserae, especially the order Galliformes which includes, without limitation, chickens, quails, ducks, geese, emus, ostriches, pheasant, and turkeys.

The term “intestinal health status” refers to the status of the gut wall structure and morphology which can be affected by, for example, infectious agents or a non-infectious cause, such as a suboptimal formulated diet. “Gut wall structure and morphology” can refer to, without limitation, epithelial damage and epithelial permeability which is characterized by a shortening of villi, a lengthening of crypts and an infiltration of inflammatory cells (such as, without limitation, CD3+ cells). The latter damage and inflammation markers can also be associated with a “severe” macroscopic appearance of the gut—compared to a “normal” appearance—when evaluated using a scoring system such as the one described by Teirlynck et al. (2011).

The phrase “poor intestinal health” refers to gut wall structure and morphology resulting from, for example, infectious agents or a non-infectious cause, such as a suboptimal formulated diet. A domesticated bird with poor intestinal health exhibits abnormal gut wall structure and morphology which is evidenced by, without limitation, one or more of epithelial damage and epithelial permeability characterized by one or more of shortening of villi, lengthening of crypts, and/or and an infiltration of inflammatory cells (such as, without limitation, CD3+cells). The latter damage and inflammation markers can also be associated with a “severe” macroscopic appearance of the gut—compared to a “normal” appearance—when evaluated using a scoring system such as the one described by Teirlynck et al. (2011).

The term “fecal sample” refers to fecal droppings from birds.

The term “intestinal content sample” can refer to intestinal content obtained from, for example, necropsy of birds. The term “intestinal content at necropsy of birds” refers to a sample taken from the content present in one or more of the gizzard, ileum, caecum or colon, such as after said bird is euthanized. In other embodiments, “intestinal content sample” can refer to the contents of the intestines as well as the intestinal tissue itself. In further embodiments, “intestinal content sample” can refer to a sample obtained via mucosal scratching.

The phrase “quantifying populations of one or more microorganism(s) in a fecal or intestinal content sample” refers to any method known to a person having ordinary skill in the art to quantify and/or identify said one or more microorganism(s) in the sample. Non-limiting examples of such methods include mass-spectrometrical methods, ELISA and Western Blotting, real-time PCR, and sequencing of microbial 16S ribosomal DNA (rDNA) genes. It should be clear that the quantification of a single microorganism might be sufficient to determine intestinal health status but that also a combination of any of about 2, 3, 4, 5, 6, 7, 8, 9 or more microorganisms can be used to determine the intestinal health status of the poultry.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting of,” as used herein, means including, and limited to, the component(s) after the term “consisting of” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

II. Methods

Provided herein are methods for determining the intestinal health status of a domesticated bird by quantifying populations of one or more microorganism(s) in a fecal and/or intestinal content sample from the bird. In one non-limiting embodiment, the microorganism(s) are selected from the Clostridiales vadinBB60 group family of microorganisms and/or a microorganism from the Peptostreptococcaceae family (e.g., Peptoclostridium difficile) of microorganisms.

Both the vadinBB60 group family and the Peptostreptococcaceae families of microorganisms are in the Clostridiales order of microorganisms and constitute a highly polyphyletic class of the phylum Firmicutes. Microbes in these families are gram positive and distinguished from the Bacilli by lacking aerobic respiration. Specifically, they are obligate anaerobes and oxygen is toxic to them (Bergey's manual of systematics of archaea and bacteria, Witman, Sup. Ed., Hoboken, N.J.: Wiley (2015); Galperin et al., 2016, Int. J. System. & Evol. Microbiol., 66:5506-13).

As described in the Examples section, when poultry were administered therapeutic levels of antibiotics to induce dysbiosis followed by a cocktail containing opportunistic bacterial pathogens as well as a coccidial cocktail, a statistically significant decrease in the population of vadinBB60 group family and Peptostreptococcaceae family microorganisms was observed in comparison to the level of these microorganisms that were found in samples obtained from healthy control animals.

Moreover, additional microorganisms were identified from the genera Brevibacterium, Brachybacterium, Ruminiclostridium, Candidatus Arthromitus, Ruminococcus (with the optional exception of Ruminococcus torques; for example, R. lactatiformans), Streptococcus, Shuttleworthia, Lachnospiraceae NK4A136 group, and Ruminococcaceae UCG-005. These microorganisms were also observed to significantly decrease in challenged birds in comparison to non-challenged control animals.

Brevibacterium is a genus of bacteria of the order Actinomycetales. They are Gram-positive soil organisms and represent the sole genus in the family Brevibacteriaceae. Representative species of Brevibacterium include, without limitation, B. acetyliticum, B. albidum, B. antiquum, B. aurantiacum, B. avium, B. casei, B. celere, B. divaricatum, B. epidermidis, B. frigoritolerans, B. halotolerans, B. immotum, B. iodinum, B. linens, B. luteolum, B. luteum, B. mcbrellneri, B. otitidis, B. oxydans, B. paucivorans, B. permense, B. picturae, B. samyangense, B. sanguinis, and B. stationis.

Brachybacterium is a genus of Gram positive, nonmotile bacteria. The cells are coccoid during the stationary phase, and irregular rods during the exponential phase. Representative species of Brachybacterium include, without limitation, B. alimentarium, B. aquaticum, B. conglomeratum, B. faecium, B. fresconis, B. ginsengisoli, B. horti, B. huguangmaarense, B. massiliense, B. muris, B. nesterenkovii, B. paraconglomeratum, B. phenoliresistens, B. rhamnosum, B. sacelli, B. saurashtrense, B. squillarum, B. tyrofermentans, and B. zhongshanense.

Ruminiclostridium are obligately anaerobic, mesophilic or moderately thermophilic, spore-forming, straight or slightly curved rods 0.5-1.5 μm×1.5-8 μm. The cells have a typical Gram-positive cell wall, although often stain Gram-negative. Produce spherical or oblong terminal spores, which results in swollen cells. Most species are motile and have polar, subpolar, or peritrichous flagella (see Yutin & Galperin, Environ Microbiol. 2013 October; 15(10): 2631-2641). When this genus was proposed, the formerly named species Clostridium thermocellum, C. aldrichii, C. alkalicellulosi, C. caenicola, C. cellobioparum, C. cellulolyticum, C. cellulosi, C. clariflavum, C. hungatei, C. josui, C. leptum, C. methylpentosum, C. papyrosolvens, C. sporosphaeroides, C. stercorarium, C. straminisolvens, C. sufflavum, C. termitidis, C. thermosuccinogenes, C. viride, Bacteroides cellulosolvens, and Eubacterium siraeum were reclassified into this genus (Yutin & Galperin, Environ Microbiol. 2013 October; 15(10): 2631-2641).

Candidatus Arthromitus is a genus of morphologically distinct bacteria found almost exclusively in terrestrial arthropods. They are gram-positive, spore-forming bacteria that possess the capability to develop into long filaments and known to intimately bind to the surface of absorptive intestinal epithelium without inducing an inflammatory response. The 16S rRNA gene sequences of picked Arthromitus filaments shows them to form a diverse but closely related group of arthropod-derived sequences within the Lachnospiraceae.

Ruminococcus is a genus of bacteria in the class Clostridia. They are anaerobic, Gram-positive gut microbes. Representative species of Ruminococcus include, without limitation, Ruminococcus albus, Ruminococcus bromii, Ruminococcus callidus, Ruminococcus flavefaciens, Ruminococcus gauvreauii, Ruminococcus gnavus, Ruminococcus lactaris, Ruminococcus obeum. In some embodiments, the Ruminococcus species does not include Ruminococcus torques.

Streptococcus is a genus of gram-positive coccus (plural cocci) or spherical bacteria that belongs to the family Streptococcaceae, within the order Lactobacillales (lactic acid bacteria), in the phylum Firmicutes. Cell division in streptococci occurs along a single axis, so as they grow, they tend to form pairs or chains that may appear bent or twisted. Representative species of Streptococcus include, without limitation Streptococcus acidominimus, S. agalactiae, S. alactolyticus, S. anginosus, S. australis, S. bovis, S. caballi, S. cameli, S. canis, S. caprae, S. castoreus, S. criceti, S. constellatus, S. cuniculi, S. danieliae, S. dentasini, S. dentiloxodontae, S. dentirousetti, S. devriesei, S. didelphis, S. downei, S. dysgalactiae, S. entericus, S. equi, S. equinus, S. ferus, S. gallinaceus, S. gallolyticus, S. gordonii, S. halichoeri, S. halotolerans, S. henryi, S. himalayensis, S. hongkongensis, S. hyointestinalis, S. hyovaginalis, S. ictalurid, S. infantarius, S. infantis, S. iniae, S. intermedius, S. lactarius, S. loxodontisalivarius, S. lutetiensis, S. macacae, S. marimammalium, S. marmotae, S. massiliensis, S. merionis, S. minor, S. milleri, S. mitis, S. moroccensis, S. mutans, S. oligofermentans, S. oxalis, S. oricebi, S. oriloxodontae, S. orisasini, S. orisratti, S. orisuis, S. ovis, S. panodentis, S. pantholopis, S. parasanguinis, S. parasuis, S. parauberis, S. peroris, S. pharynges, S. phocae, S. pluranimalium, S. plurextorum, S. pneumoniae, S. porci, S. porcinus, S. porcorum, S. pseudopneumoniae, S. pseudoporcinus, S. pyogenes, S. ratti, S. rifensis, S. rubneri, S. rupicaprae, S. salivarius, S. saliviloxodontae, S. sanguinis, S. sinensis, S. sobrinus, S. suis, S. tangierensis, S. thoraltensis, S. troglodytae, S. troglodytidis, S. tigurinus, S. thermophilus, S. uberis, S. urinalis, S. ursoris, S. vestibularis, S. viridans, and S. zooepidemicus.

Shuttleworthia is a Gram-positive, non-spore-forming, obligately anaerobic and non-motile bacterial genus from the family of Lachnospiraceae with one known species (Shuttleworthia satelles).

The Lachnospiraceae NK4A136 group are a genus of bacteria in the family Lachnospiraceae and in the order of Clostridiales which occur in the human and mammal gut microbiota. All species of this genus are anaerobic.

Ruminococcaceae UCG-005 is a genus of bacteria in the family Ruminococcaceae which is in the class Clostridia. All Ruminococcaceae UCG-005 species are obligate anaerobes. However, members of the family have diverse shapes, with some rod-shaped and others cocci.

In additional embodiments, the method can also include identifying (i.e. detecting) and quantifying one or more microorganism from an intestinal content sample from the genus Defluviitaleaceae UCG-011, a microorganism from the genus Lachnoclostridium, or a microorganism from the Ruminococcus torques group. In this embodiment, a decreased population of one or more microorganism(s) of these genera in a sample obtained from the caecum is an indicator of poor intestinal health, when compared to the level found in caecum samples of non-challenged healthy control animals. However, an increased population of one or more microorganism(s) of these genera in a sample obtained from the colon is an indicator of poor intestinal health, when compared to the level found in colon samples of non-challenged healthy control animals.

Defluviitaleaceae UCG-011 is a genus of bacteria in the family Defluviitaleaceae, a family in the order Clostridiales. Lachnoclostridium is a genus of bacteria in the family Lachnospiraceae, a family in the order Clostridiales. Ruminococcus torques is a species of bacteria in the Ruminococcus genus.

In yet further embodiments, the method can also include identifying (i.e. detecting) and quantifying one or more microorganism from an intestinal content sample from the genus Lactobacillus. In this embodiment, a decreased population of one or more microorganism(s) of these genera in a sample obtained from the colon is an indicator of poor intestinal health, when compared to the level found in colon samples of non-challenged healthy control animals. However, an increased population of one or more microorganism(s) of these genera in a sample obtained from the caecum is an indicator of poor intestinal health, when compared to the level found in caecum samples of non-challenged healthy control animals.

Lactobacillus is a genus of Gram-positive, facultative anaerobic or microaerophilic, rod-shaped, non-spore-forming bacteria. They are a major part of the lactic acid bacteria group (i.e., they convert sugars to lactic acid). Representative species of Lactobacillus include, without limitation Lactobacillus acetotolerans, L. acidifarinaegenenc, L. acidipiscis, L. acidophilus, L. agilis, L. algidus, L. alimentarius, L. allii, L. alvei, L. alvi, L. amylolyticus, L. amylophilus, L. amylotrophicus, L. amylovorus, L. animalis, L. animate, L. antri, L. apinorum, L. apis, L. apodemi, L. aquaticus, L. aviarius, L. backii, L. bambusae, L. bifermentans, L. bombi, L. bombicola, L. brantae, L. brevis, L. brevisimilis, L. buchneri, L. cacaonum, L. camelliae, L. capillatus, L. casei, L. chiayiensis, L. paracasei, L. zeae, L. catenefornis, L. caviae, L. cerevisiae, L. ceti, L. coleohominis, L. colini, L. collinoides, L. composti, L. concavus, L. coryniformis, L. crispatus, L. crustorum, L. curieae, L. curtus, L. curvatus, L. delbrueckii, L. dextrinicus, L. diolivorans, L. equi, L. equicursoris, L. equigenerosi, L. fabifermentans, L. faecis, L. faeni, L. farciminis, L. farraginis, L. fermentum, L. floricola, L. forum, L. formosensis, L. fornicalis, L. fructivorens, L. frumenti, L. fuchuensis, L. furfuricola, L. futsaii, L. gallinarum, L. gasseri, L. gastricus, L. ghanensis, L. gigeriorum, L. ginsenosidimutans, L. gorillae, L. graminis, L. guizhouensis, L. halophilus, L. hammesii, L. hamsteri, L. harbinensis, L. hayakitensis, L. heilongjiangensis, L. helsingborgensis, L. helveticus, L. herbarum, L. heterohiochii, L. hilgardii, L. hokkaidonensis, L. hominis, L. homohiochii, L. hordei, L. iatae, L. iners, L. ingluviei, L. insectis, L. insicii, L. intermedius, L. intestinalis, L. iwatensis, L. ixorae, L. japonicus, L. jensenii, L. johnsonii, L. kalixensis, L. kefiranofacien, L. kefiri, L. kimbladii, L. kimchicus, L. kimchiensis, L. kisonensis, L. kitasatonis, L. koreensis, L. kosoi, L. kullabergensis, L. kunkeei, L. larvae, L. leichmannii, L. letivazi, L. lindneri, L. malefermentans, L. mali, L. manihotivorans, L. mellifer, L. mellis, L. melliventris, L. metriopterae, L. micheneri, L. mindensis, L. mixtipabuli, L. mobilis, L. modestisalitolerans, L. mucosae, L. mudanjiangensis, L. murinus, L. musae, L. nagelii, L. namurensis, L. nantensis, L. nasuensis, L. nenjiangensis, L. nodensis, L. nuruki, L. odoratitofui, L. oeni, L. oligofermentans, L. oris, L. oryzae, L. otakiensis, L. ozensis, L. panis, L. panisapium, L. pantheris, L. parabrevis, L. parabuchneri, L. paracollinoides, L. parafarraginis, L. paragasseri, L. parakefiri, L. paralimentarius, L. paraplantarum, L. pasteurii, L. paucivorans, L. pentosiphilus, L. pentosus, L. perolens, L. plajomi, L. plantarum, L. pobuzihii, L. pontis, L. porci, L. porcinae, L. psittaci, L. quenuiae, L. raoultii, L. rapi, L. rennanquilfy, L. rennini, L. reuteri, L. rhamnosus, L. rodentium, L. rogosae, L. rossiae, L. ruminis, L. saerimneri, L. sakei, L. salivarius, L. sanfranciscensis, L. saniviri, L. satsumensis, L. secaliphilus, L. selangorensis, L. senioris, L. senmaizukei, L. sharpeae, L. shenzhenensis, L. sicerae, L. silage, L. silagincola, L. siliginis, L. similis, L. songhuajiangensis, L. spicheri, L. sucicola, L. suebicus, L. sunkii, L. taiwanensis, L. terrae, L. thailandensis, L. timberlakei, L. timonensis, L. tucceti, L. ultunensis, L. uvarum, L. vaccinostercus, L. vaginalis, L. vermiforme, L. versmoldensis, L. vespulae, L. vini, L. wasatchensis, L. xiangfangensis, L. yonginensis, and L. zymae.

In another embodiments the method can also include identifying (i.e. detecting) and quantifying one or more microorganism from a fecal and/or intestinal content sample from a microorganism from the phylum Tenericutes and/or Firmicutes; a microorganism from the class Mollicutes RF39, Erysipelotrichales, Clostridiales, and/or Micrococcales; a microorganism from the family Streptococcaceae, Defluviitaleaceae, Christensenellaceae, Erysipelotrichaceae, Lachnospiraceae, Ruminococcaceae, Dermabacteraceae, Brevibacteriaceae, and/or Dietziaceae; and/or a microorganism from the genus Roseburia, Harryflintia, Ruminococcaceae UCG-009, Coprococcus, Ruminococcaceae UCG-010, Ruminococcus, Christensenellaceae R-7 group, Erysipelatoclostridium, Ruminococcaceae NK4A214 group, Negativibacillus, Oscillibacter, Butyricicoccus, and/or Eisenbergiella. In this embodiment, a decreased population of one or more microorganism(s) of these phyla, classes, families, or genera when compared to the level found in fecal or intestinal content samples of healthy control animals, is an indicator of poor intestinal health. The intestinal content sample can be derived from ileum and/or caecum.

Additional embodiments of the method include identifying (i.e. detecting) and quantifying one or more microorganism from a fecal and/or intestinal content sample from a microorganism from the phylum Verrucomicrobia and/or Bacteroidetes; a microorganism from the class Coriobacteriales, Verrucomicrobiales and/or Bacteroidales; a microorganism from the family Eggerthellaceae, Akkermansiaceae, Lactobacillaceae, and/or Clostridiaceae; and/or a microorganism from the genus Eggerthella, and/or Akkermansia. In this embodiment, an increased population of one or more microorganism(s) of these phyla, classes, families, or genera when compared to the level found in fecal or intestinal content samples of healthy control animals, is an indicator of poor intestinal health. The intestinal content sample can be derived from ileum and/or caecum.

Alternative embodiments include identifying (i.e. detecting) and quantifying populations of one or more microorganism(s) in a fecal and/or intestinal content sample from the order Rhodospirillales; and/or from the genus Helicobacter, Staphylococcus, Jeotgalicoccus, Ruminococcus, Marvinbryantia, Ruminococcaceae UCG-013, Enterococcus, Corynebacterium, and/or Subdoligranulum. In this embodiment, a decreased population of one or more microorganism(s) of this order or genera when compared to the level found in fecal or intestinal content samples of healthy control animals, is an indicator of poor intestinal health. The intestinal content sample can be derived from colon and/or caecum.

In another embodiment, the method further includes identifying (i.e. detecting) and quantifying populations of one or more microorganism(s) in a fecal and/or intestinal content sample from the genus Firmicutes, Anaerofilum, Intestinimonas, Fournierella, Barnesiella, Barnesiella, Bifidobacterium, Tyzzerella, Clostridium sensu stricto, and/or Escherichia-Shigella. In this embodiment, an increased population of one or more microorganism(s) of these genera when compared to the level found in fecal or intestinal content samples of healthy control animals, is an indicator of poor intestinal health. The intestinal content sample can be derived from colon and/or caecum.

Intestinal health can be determined in accordance with any number of means known in the art including, without limitation, measuring villus length; measuring villus-to crypt ratio; measuring T-lymphocyte infiltration in villi; and/or scoring the macroscopic gut appearance of the birds. Methods for determining intestinal health are described in detail in the Examples section. Similarly, quantification and identification of microorganisms can be conducted using any means known in the art, such as, but not limited to antibody based assays (for example, ELISA or Western Blot) or a PCR-based assay (for example, sequencing of the microbial 16S ribosomal DNA (rDNA) gene).

In further embodiments, the method additionally can include identifying (i.e. detecting) and quantifying one or more metabolite(s) in a fecal and/or intestinal content sample from the bird selected from the group consisting of linoleyl carnitine, linalool, 3-[(9Z)-9-octadecenoyloxy]-4-(trimethylammonio)butanoate, (−)-trans-methyl dihydrojasmonate, icomucret, 1,3-dioctanoylglycerol, ethyl 2-nonynoate, 4-aminobutyrate, 2-amino-isobutyrate, D-alpha-aminobutyrate, cadaverine, putrescine, uracil, hypoxanthine, D-alanine, sarcosine, methional, hexanal, malondialdehyde L-alanine, and acetylcarnitine. In this embodiment, an increased level of the one or more metabolite(s), when compared to the level found in fecal or intestinal content samples of healthy control animals, is an indicator of poor intestinal health. Any method known in the art can be used to quantify and identify the metabolites, such as, without limitation, antibody based assays (for example, ELISA or Western Blot), HPLC, or mass spec.

In another embodiment, the method further includes quantifying one or more metabolite(s) in a fecal and/or intestinal content sample from the bird selected from the group consisting of 5-(2-carboxyethyl)-2-hydroxyphenyl beta-D-glucopyranosiduronic acid, 4,15-Diacetoxy-3-hydroxy-12,13-epoxytrichothec-9-en-8-yl3-hydroxy-3-methylbutanoate, scoparone, asp-leu, ethyl benzoylacetate, L-(+)-glutamine, 1-allyl-2,3,4,5-tetramethoxybenzene, (DL)-3-O-methyldopa, dictyoquinazol A, 1-(3-furyl)-7-hydroxy-4,8-dimethyl-1,6-nonanedione methyl 3,4,5-trimethoxycinnamate, butylparaben, aspartic acid, L-arginine, glutamic acid, L-pyroglutamic acid, L-glutamine, L-histidine, glycine, (−)-beta-pineen, L-asparagine, L-homoserine, L-serine, L-threonine, L-prdine, L-tyrosine, L-leucine, dopamine, taurocholic acid, tryptamine, tauroursodeoxychdic acid, glycoursodeoxycholic acid, ursodeoxycholic acid, cholic acid, nonanal, 3-methyl-2-butenal, DL-glyceraldehyde, allantoin, nicotinic acid, N-acetylglucosamine, spermidine, (dimethylamino)acetonitrile, glycoursodeoxycholic acid, tauroursodeoxycholic acid, cortisol, and heptanal. In this embodiment, a decreased level of said one or more metabolite(s) in said fecal or intestinal content sample, when compared to the level found in fecal or intestinal content samples of healthy control animals, is an indicator of poor intestinal health. Any method known in the art can be used to quantify and identify the metabolites, such as, without limitation, antibody based assays (for example, ELISA or Western Blot), HPLC, or mass spec.

The invention can be further understood by reference to the following examples, which are provided by way of illustration and are not meant to be limiting.

### EXAMPLES

### Example 1

### Assays

In the following examples, various assays were used as set forth below for ease in reading. Any deviations from the protocols provided below are indicated in the relevant sections. In these experiments, a spectrophotometer was used to measure the absorbance of the products formed after the completion of the reactions.

Histology: The duodenal loop was fixated in 4% formaldehyde for 24 hours, dehydrated in xylene and embedded in paraffin. Sections of 4 μm were cut using a microtome (Microme HM360, Thermo Scientific) and were processed as described by De Maesschalck et al. (2015). Villus length and crypt depth in the duodenum were determined by random measurement of twelve villi per intestinal segment using standard light microscopy (Leica DM LB2 Digita) and a computer based image analysis program, LAS V4.1 (Leica Application Suite V4, Germany). Afterwards the villus-to-crypt ratio was calculated. Antigen retrieval was performed on 4 μm duodenal sections with a pressure cooker in citrate buffer (10 mM, pH 6). Slides were rinsed with washing buffer (Dako kit, K4011) and blocked with peroxidase reagent (Dako, 52023) for 5 minutes. Slides were rinsed with Aquadest and Dako washing buffer before incubation with anti-CD3 primary antibodies (Dako CD3, A0452) for 30 minutes at room temperature diluted 1:100 in antibody diluent (Dako, S3022). After rinsing again with washing buffer, slides were incubated with labelled polymer-HRP anti-rabbit (Envision+ System-HRP, K4011) for 30 minutes at room temperature. Before adding di-amino-benzidine (DAB+) substrate and DAB+ chromogen (Dako kit, K4011) for 5 minutes, slides were rinsed 2 times with washing buffer. To stop the staining, the slides were rinsed with Aquadest, dehydrated using the Shandon Varistain-Gemini Automated Slide Stainer and counterstained with hematoxylin for 10 seconds. The slides were analyzed with Leica DM LB2 Digital and a computer based image analysis program LAS V4.1 (Leica Application Suite V4, Germany) to measure CD3 positive area on a total area of 3 mm² which represents T-lymphocyte infiltration in approximately 10 villi per section.

DNA Extraction: DNA was extracted from caecum and colon content using the hexadecyltrimethylammonium bromide (CTAB) method as described previously (28, 29). To 100 mg of intestinal content, 0.5 g unwashed glass beads (Sigma-Aldrich, St. Louis, Mo.), 0.5 ml CTAB buffer (5% [wt/vol] hexadecyltrimethylammonium bromide, 0.35 M NaCl, 120 mM K2HPO4) and 0.5 ml phenol-chloroform-isoamyl alcohol mixture (25:24:1) (Sigma-Aldrich, St. Louis, Mo.) were added, followed by homogenization in a 2-ml destruction tube. The samples were shaken 6 times for 30 s each using a beadbeater (MagnaLyser; Roche, Basel, Switzerland) at 6,000 rpm with 30 s between shakings. After centrifugation (10 min, 8000 rpm), 300 μl of the supernatant was transferred to a new tube. The rest of the tube content was reextracted with 250 μl CTAB buffer and again homogenized with a beadbeater. The samples were centrifuged for 10 min at 8,000 rpm, and 300 μl supernatant was added to the first 300 μl supernatant. The phenol was removed by adding an equal volume of chloroform-isoamyl alcohol (24:1) (Sigma-Aldrich, St. Louis, Mo.) and performing a short spin. The aqueous phase was transferred to a new tube. The nucleic acids were precipitated with two volumes of polyethylene glycol (PEG) 6000 solution (30% [wt/vol] PEB, 1.6 M NaCl) for 2 h at room temperature. After centrifugation (20 min, 13,000 rpm), the pellet was rinsed with 1 ml of ice-cold 70% (vol/vol) ethanol. The pellet was dried and resuspended in 100 μl RNA-free water (VWR, Leuven, Belgium). The quality and the concentration of the DNA was examined spectrophotometrically (NanoDrop, Thermo Scientific, Waltham, Mass., USA).

Library Prep: DNA was extracted from caecum and colon content using the hexadecyltrimethylammonium bromide (CTAB) method as described previously (28, 29). To 100 mg of intestinal content, 0.5 g unwashed glass beads (Sigma-Aldrich, St. Louis, Mo.), 0.5 ml CTAB buffer (5% [wt/vol] hexadecyltrimethylammonium bromide, 0.35 M NaCl, 120 mM K2HPO4) and 0.5 ml phenol-chloroform-isoamyl alcohol mixture (25:24:1) (Sigma-Aldrich, St. Louis, Mo.) were added, followed by homogenization in a 2-ml destruction tube. The samples were shaken 6 times for 30 s each using a beadbeater (MagnaLyser; Roche, Basel, Switzerland) at 6,000 rpm with 30 s between shakings. After centrifugation (10 min, 8000 rpm), 300 μl of the supernatant was transferred to a new tube. The rest of the tube content was reextracted with 250 CTAB buffer and again homogenized with a beadbeater. The samples were centrifuged for 10 min at 8,000 rpm, and 300 μl supernatant was added to the first 300 μl supernatant. The phenol was removed by adding an equal volume of chloroform-isoamyl alcohol (24:1) (Sigma-Aldrich, St. Louis, Mo.) and performing a short spin. The aqueous phase was transferred to a new tube. The nucleic acids were precipitated with two volumes of polyethylene glycol (PEG) 6000 solution (30% [wt/vol] PEB, 1.6 M NaCl) for 2 h at room temperature. After centrifugation (20 min, 13,000 rpm), the pellet was rinsed with 1 ml of ice-cold 70% (vol/vol) ethanol. The pellet was dried and resuspended in 100 μl RNA-free water (VWR, Leuven, Belgium). The quality and the concentration of the DNA was examined spectrophotometrically (NanoDrop, Thermo Scientific, Waltham, Mass., USA).

To identify the taxonomic groups in the ileal, caecal and colon microbiota of the chickens, the V3-V4 hypervariable region of 16s rRNA gene was amplified using the gene-specific primers S-D-Bact-0341-b-S-17 (5′-TCGTCGGCAGCGTCAGATGTGTATAAGAGACAGCCTACGGGNGGCWGCAG-3′) and S-D-Bact-0785-a-A-21 (5′-GTCTCGTGGGCTCGGAGATGTGTATAAGAGACAGGACTACHVGGGTATCTAATCC-3′) (Klindworth, et al., 2013). Each 25 μl PCR reaction contained 2.5 μl DNA (˜5 ng/μl), 0.2 μM of each of the primers and 12.5 μl×KAPA HiFi HotStart ReadyMix (Kapa Biosystems, Wilmington, Mass., USA). The PCR amplification consisted of initial denaturation at 95° C. for 3 min, followed by 25 cycles of 95° C. for 30 s, 55° C. for 30 s, 72° C. for 30 s and a final extension at 72° C. for 5 min. The PCR products were purified using CleanNGS beads (CleanNA, Waddinxveen, The Netherlands). The DNA quantity and quality was analyzed spectrophotometrically (NanoDrop) and by agarose gel electrophoresis. A second PCR step was used to attach dual indices and Illumina sequencing adapters in a 50 μl reaction volume containing 5 μl of purified PCR product, 2×KAPA HiFi HotStart ReadyMix (25 μl) and 0.5 μM primers. The PCR conditions were the same as the first PCR with the number of cycles reduced to 8. The final PCR products were purified and the concentration was determined using the Quantus double-stranded DNA assay (Promega, Madison, Wis., USA). The final barcoded libraries were combined to an equimolar 5 nM pool and sequenced with 30% PhiX spike-in using the Illumina MiSeq v3 technology (2×300 bp, paired-end) at the Oklahoma Medical Research Center (Oklahoma City, Okla., USA) for samples from trial 1 and at Macrogen (Seoul, Korea) for samples from trial 2.

Bioinformatics and statistical analysis of 16S rRNA gene amplicon data: Demultiplexing of the amplicon dataset and deletion of the barcodes was done by the sequencing provider. Quality of the raw sequence data was checked with the FastQC quality-control tool (Babraham Bioinformatics, Cambridge, United Kingdom; http://www.bioinformatics.babraham.ac.uk/projects/fastqc/) followed by initial quality filtering using Trimmomatic v0.38 by cutting reads with an average quality per base below 15 using a 4-base sliding window and discarding reads with a minimum length of 200 bp (Bolger, et al., 2014). The paired-end sequences were assembled and primers were removed using PANDAseq (Masella, et al., 2012), with a quality threshold of 0.9 and length cut-off values for the merged sequences between 390 and 430 bp. Chimeric sequences were removed using UCHIME (Edgar, et al., 2011). Open-reference operational taxonomic unit (OTU) picking was performed at 97% sequence similarity using USEARCH (v6.1) and converted to an OTU table (Edgar, 2010). OTU taxonomy was assigned against the Silva database (v128, clustered at 97% identity) (Quast, et al., 2013) using the PyNast algorithm with QIIME (v1.9.1) default parameters (Caporaso, et al., 2010). OTUs with a total abundance below 0.01% of the total sequences were discarded (Bokulich, et al., 2013), resulting in an average of approximately 26920 reads per sample. Alpha rarefaction curves were generated using the QIIME “alpha_rarefaction.py” script and in trial 1 a subsampling depth of 15 000 reads was selected. One ileal sample from the control group was excluded from further analysis due to insufficient sequencing depth. Any sequences of mitochondrial or chloroplastic origins were removed. In trial 2 a subsampling depth of 9900 reads was selected. One caecal sample from the control group and one caecal sample from the challenge group was excluded from further analysis due to insufficient sequencing depth. Any sequences of mitochondrial or chloroplastic origins were removed.

Further analysis of alpha diversity (Observed OTUs, Chao1 richness estimator and Shannon diversity estimator) and beta diversity (Bray-Curtis dissimilarities) were performed using the phyloseq (McMurdie and Holmes, 2013) pipeline in R (v3.4.3). Normality of the alpha diversity data was tested using the Shapiro-Wilk test. A t-test was used for normal distributed data, whereas the Mann-Whitney U test was used for not normal distributed data. Differences in beta diversity were examined using the anosim function from the vegan package. Differences in relative abundance at the phylum level were assessed using the two-sided Welch t-test from the mt wrapper in phyloseq, with the P-value adjusted for multiple hypothesis testing using the Benjamini-Hochberg method. To detect differentially abundant taxa between the control and challenge group, both DESeq2 analysis and Linear Discriminant Analysis (LDA) Effect Size (LEfSe) analysis were used. DESeq2 was applied on the non-rarified community composition data for either caecal or ileal communities (Love, et al., 2014). Significant differences were obtained using a Wald test followed by a Benjamini-Hochberg multiple hypothesis correction. LEfSe analysis was performed on Genus level using the LEfSe wrapper “koeken.py” with an ANOVA p-value<0.05 and logarithmic LDA score threshold of 2.0 (Segata et al., 2011). The correlation of bacterial taxa with different bird characteristics (body weight, dysbiosis score, coccidiosis score, or histological parameters (crypt depth, villus length, villus-to-crypt ratio or CD3 area percentage)) was assessed using the QIIME “observation_metadata_correlation.py” script. For each group (control or challenge) and each intestinal segment (ileum, caecum or colon), the Spearman correlation coefficient was calculated using the relative abundance of all families and genera versus each bird parameter. The resulting p-values were corrected by the Benjamini-Hochberg FDR procedure for multiple comparisons. For all tests, a P-value<0.05 was considered significant.

Metabolomics: After freeze-drying of the colon and caecum content, 100 mg was weighted and resuspended in 2 ml ice cold 80% methanol. L-alanine d3 was used as internal standard. Herefore 25 μl of 100 ng/μl stock was added. Following vortexing (1 min) and centrifugation (10 min 9000 rpm) the supernatant was filter sterilized (0.45 μm) and diluted (1:3) with ultra-pure water. After vortexing (15 s) the filtrate was transferred into LC-MS vials.

An ultrahigh performance liquid chromatography hyphenated to Orbitrap HRMS (UHPLC-HRMS) was used for the chromatographic separation of the gastrointestinal (GIT)-derived metabolites using a Hypersil Gold column (1.9 μm, 100×2.1 mm) (Thermo Fisher Scientific, San-Francisco, USA) kept at 45° C. As binary solvent system, ultrapure water (A) and acetonitrile (B) both acidified with 0.1% formic acid were used and pumped at a flow rate of 400 μL min−1. The linear gradient program with the following proportions (v/v) of solvent A was applied: 0-1.5 min at 98%, 1.5-7.0 min from 98% to 75%, 7.0-8.0 min from 75% to 40%, 8.0-12.0 min from 40% to 5%, 12.0-14.0 min at 5%, 14.0-14.1 min from 5% to 98%, followed by 4.0 min of reequilibration. The injection volume of each sample was 10 μL.

HRMS analysis was performed on an Exactive stand-alone benchtop Orbitrap mass spectrometer (Thermo Fisher Scientific, San Jose, Calif., USA), equipped with a heated electrospray ionization source (HESI), operating in polarity switching mode. Ionization source working parameters were optimized and were set to a sheath, auxiliary, and sweep gas of 50, 25, and 5 arbitrary units (au), respectively, heater and capillary temperature of 350 and 250° C., and tube lens, skimmer, capillary, and spray voltage of 60 V, 20 V, 90 V, and 5 kV (±), respectively. A scan range of m/z 50-800 was chosen, and the resolution was set at 100 000 fwhm at 1 Hz. The automatic gain control (AGC) target was set at balanced (1×106 ions) with a maximum injection time of 50 ms.

Before and after analysis of samples, a standard mixture of 291 target analytes, with a concentration of 5 ng mL was injected to check the operational conditions of the device. To adjust for instrumental fluctuations, quality control (QC) samples (a pool of samples made from the biological test samples to be studied) were included. They were implemented at the beginning of the analytical run to stabilize the system and at the end of the sequence run for signal corrections within analytical batches. Targeted data processing was carried out with Xcalibur 3.0 software (Thermo Fisher Scientific, San Jose, Calif., USA), whereby compounds were identified based on their m/z-value, C-isotope profile, and retention time relative to that of the internal standard.

For untargeted data interpretation, the software package Sieve™ 2.2 (Thermo Fisher Scientific, San Jose, Calif., USA) was used to achieve automated peak extraction, peak alignment, deconvolution, and noise removal. This differential analysis was performed separately for the negative and positive ionization mode. As major parameters, a minimum peak intensity of 500 000 a.u., retention time width of 0.3 min, and mass window of 6 ppm were employed for feature extraction, with retention time, m/z-value and signal intensity as main feature descriptors. Normalization of the data set using the QC samples was performed to take instrumental drift into account.

Outputs of the targeted and untargeted data preprocessing were subjected to multivariate statistical, which was realized using Simca™ 14.1 software (Umetrics AB, Umea, Sweden). Principal component analysis (PCA) was performed for data exploration, to display the differentiation between the obtained fingerprints and potential outliers. This was followed by OPLS-DA to establish predictive models, which were validated by evaluating some quality parameters (R² (X) and Q² (Y), permutation testing (n ¼ 100), and cross-validated ANOVA (CV-ANOVA) (p-value<0.05).

### Example 2

### Identification of Microbial Biomarkers for Intestinal Health in Ilium and Caecum

A total of 360 day-old broilers (Ross 308) were obtained from a local hatchery and housed in floor pens on wooden shavings. Throughout the study, feed and drinking water were provided ad libitum. The broilers were randomly assigned to two treatment groups, a control and challenge group (9 pens per treatment and 20 broilers per pen). All animals were fed a commercial feed till day 12 and the feed was switched to a wheat (57.5%) based diet supplemented with 5% rye. From day 12 to 18, all animals from the challenge group received 10 mg florfenicol and 10 mg enrofloxacin per kg body weight via the drinking water daily, to induce substantial changes in the gut microbial community. After the antibiotic treatment, 1 ml of a bacterial cocktail consisting of 10⁹ cfu Escherichia coli (G.78.71), 10¹⁰ cfu Enterococcus sp. (G.78.62), 10⁹ cfu Lactobacillus salivarius (LMG22873), 10⁸ cfu Lactobacillus crispatus (LMG49479), 10⁸ cfu Clostridium perfringens (netB-) (D.39.61) and 10⁸ cfu Ruminococcus gnavus (LMG27713) was given daily by oral gavage from day 19 till 21. On day 20, the animals were administered a coccidial challenge consisting of different Eimeria sp., namely 60.000 oocysts of E. acervulina and 30.000 oocysts E. maxima. At day 26, the birds were weighed and 3 birds per pen were euthanized. The duodenal loop was sampled for histological examination and content from ileum and ceacum was collected DNA extraction.

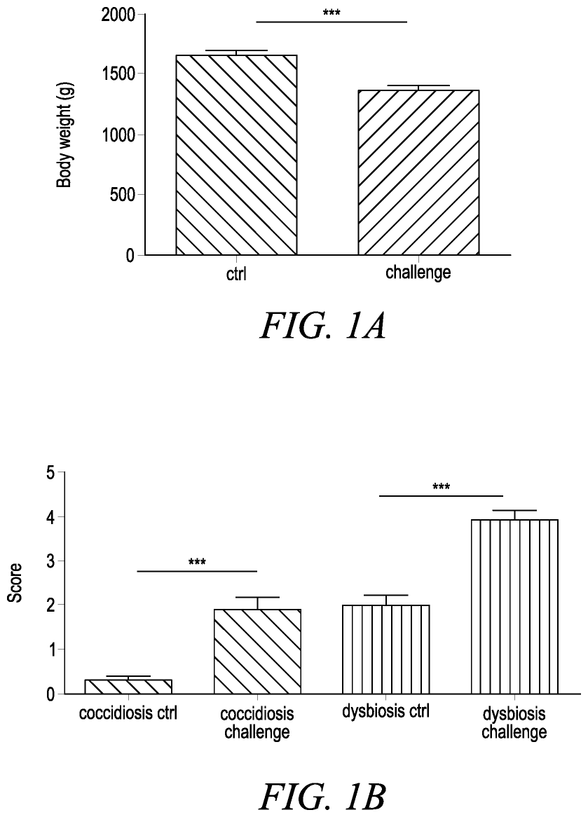

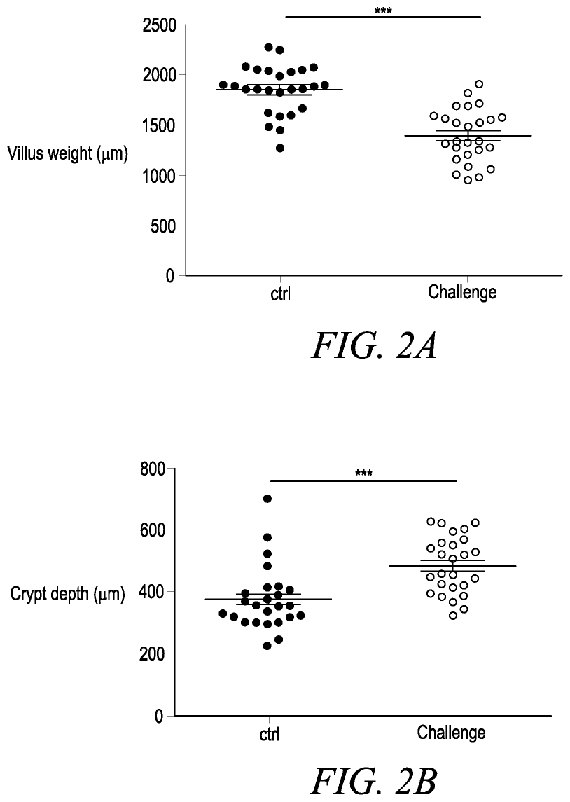

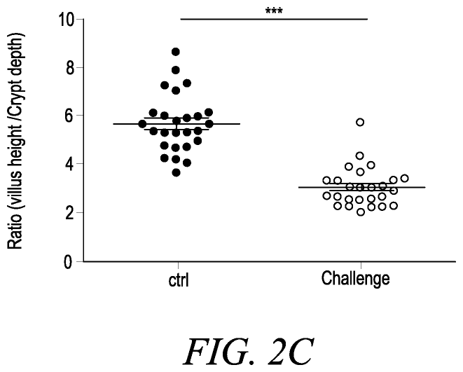

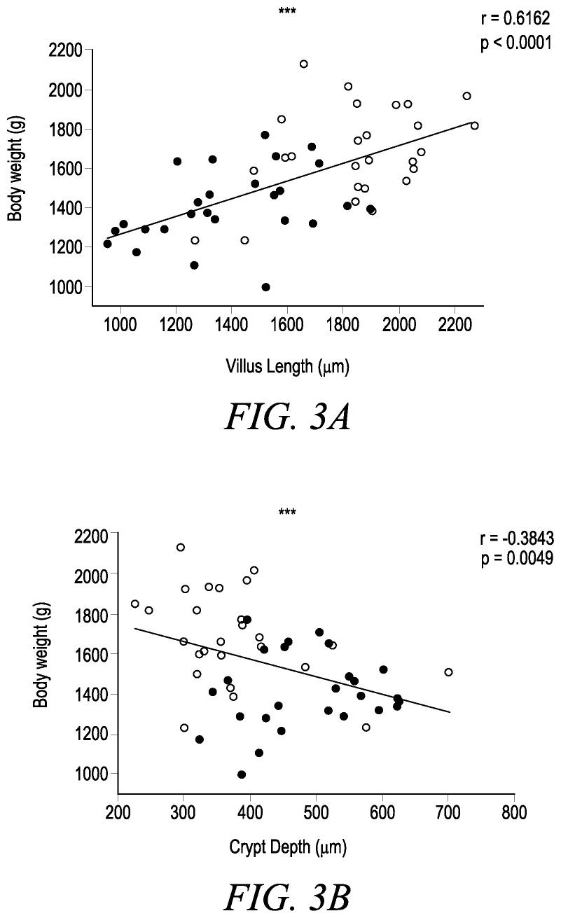

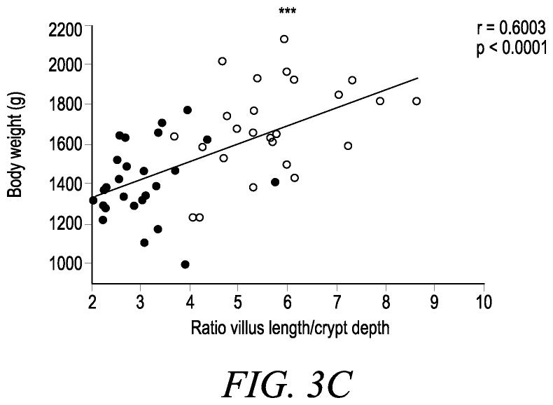

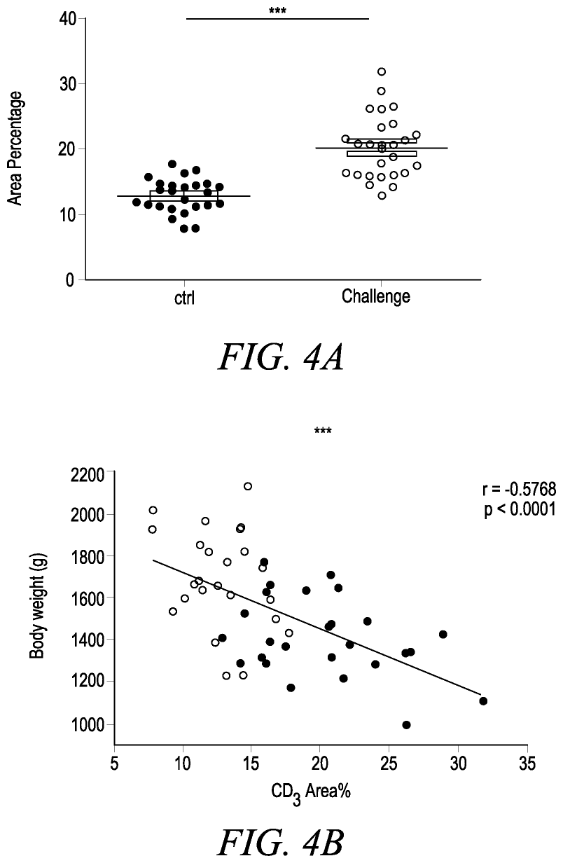

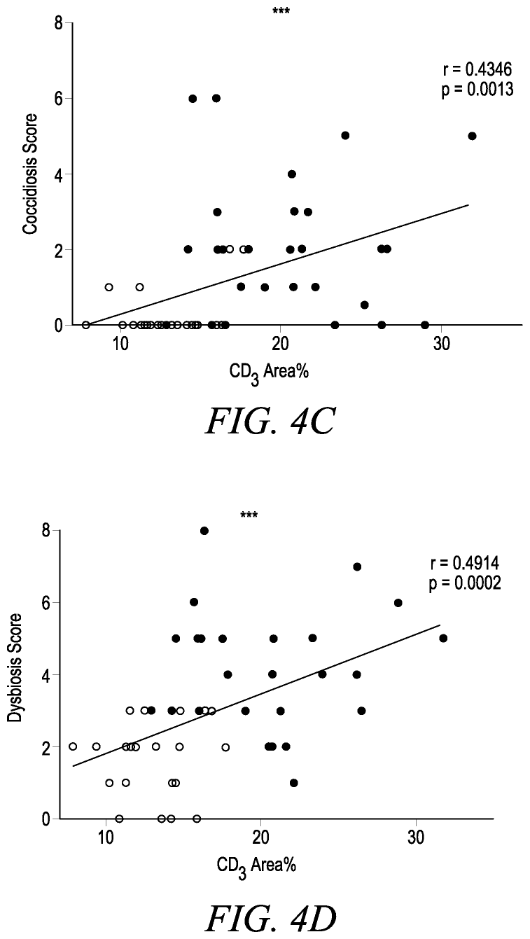

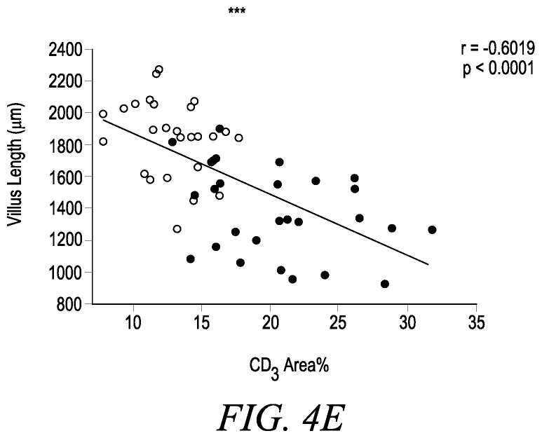

Challenged birds exhibited significant body weight reductions (FIG. 1A) as well as increased dysbiosis and coccidiosis score (FIG. 1B) each performed blindly according to De Gussem (2010; “Macroscopic scoring system for bacterialenteritis in broiler chickens and turkeys;” In WVPA Meeting (2010), Merelbeke, Belgium) and Johnson & Reid (1970; Exp. Parasitol. 28:30-36) the disclosures of which are incorporated herein, respectively. Histological evaluation revealed that challenged birds had significantly decreased villus length (FIG. 2A) and increased crypt depth (FIG. 2B; see also FIG. 2C). In particular, decreased villus length and increased crypt depth were both associated with decreased bird body weight (FIG. 3A, FIG. 3B, and FIG. 3C). Moreover, challenged birds exhibited significantly increased intestinal immune cell infiltration relative to control animals (FIG. 4A) which was correlated with decreased body weight (FIG. 4B), increased coccidiosis and dysbiosis score (FIG. 4C and FIG. 4D), and villus length (FIG. 4E). Overall, these data suggest that challenged animals exhibited significantly decreased weight and other morphological and histological symptoms associated with intestinal dysbiosis and coccidiosis.

Statistical analysis of 16S rRNA gene amplicon data was used to identify the taxonomic groups of bacteria in the ileal and caecal microbiota of control and challenged chickens as well as statistically significant changes in their populations following challenge. The results are shown in Table 1.

TABLE 1
Microbiome changes in challenged birds in ileum and caecum

| | | Ileum | | | Caecum | | |
|---|---|---|---|---|---|---|---|
| Taxa | Bacteria | Control | Challenge | p value | Control | Challenge | p value |
| Phylum | Tenericutes | | | | 0.32 | 0.06 | 0.001 |
| Phylum | Verrucomicrobia | 0.14 | 0.43 | 0.0404 | 0.72 | 4.23 | <0.0001 |
| Phylum | Bacteroidetes | | | | 17.90 | 27.34 | 0.013 |
| Phylum | Firmicutes | | | | 71.70 | 62.25 | 0.007 |
| Class | Coriobacteriia | | | | 0.13 | 0.23 | 0.004 |
| Class | Mollicutes | | | | 0.32 | 0.06 | 0.001 |
| Class | Erysipelotrichia | | | | 0.48 | 0.32 | 0.025 |
| Class | Verrucomicrobiae | 0.14 | 0.43 | 0.0404 | 0.72 | 4.23 | <0.0001 |
| Class | Bacilli | | | | 13.30 | 20.97 | 0.045 |
| Class | Bacteroidia | | | | 17.90 | 27.34 | 0.013 |
| Class | Clostridia | | | | 57.90 | 40.95 | <0.0001 |
| Order | Mollicutes RF39 | | | | 0.16 | 0.04 | 0.001 |
| Order | Coriobacteriales | | | | 0.13 | 0.23 | 0.004 |
| Order | Erysipelotrichales | | | | 0.48 | 0.32 | 0.025 |
| Order | Verrucomicrobiales | 0.14 | 0.43 | 0.0404 | 0.72 | 4.23 | <0.0001 |
| Order | Bacteroidales | | | | 17.90 | 27.34 | 0.013 |
| Order | Clostridiales | | | | 57.90 | 40.95 | <0.0001 |
| Order | Micrococcales | 0.22 | 0.02 | 0.0009 | | | |
| Family | Clostridiales vadinBB60 | | | | 7.04 | 2.54 | 0.001 |
| | group | | | | | | |
| Family | Peptostreptococcaceae | 0.52 | 0.00 | 0.0024 | 0.02 | 0.00 | 0.000 |
| Family | Streptococcaceae | | | | 0.06 | 0.00 | 0.038 |
| Family | Family XIII | | | | 0.10 | 0.03 | <0.0001 |
| Family | Defluviitaleaceae | | | | 0.13 | 0.03 | 0.001 |
| Family | Eggerthellaceae | | | | 0.13 | 0.23 | 0.004 |
| Family | Christensenellaceae | | | | 0.35 | 0.20 | 0.029 |
| Family | Erysipelotrichaceae | | | | 0.48 | 0.32 | 0.025 |
| Family | Akkermansiaceae | 0.14 | 0.43 | 0.0404 | 0.72 | 4.23 | <0.0001 |
| Family | Lachnospiraceae | | | | 14.32 | 10.16 | 0.001 |
| Family | Lactobacillaceae | | | | 13.19 | 20.88 | 0.051 |
| Family | Ruminococcaceae | | | | 35.93 | 27.48 | 0.014 |
| Family | Dermabacteraceae | 0.10 | 0.01 | 0.0016 | | | |
| Family | Clostridiaceae 1 | 1.86 | 2.56 | 0.003 | | | |
| Family | Brevibacteriaceae | 0.13 | 0.01 | 0.0024 | | | |
| Family | Dietziaceae | 0.07 | 0.02 | 0.0442 | | | |
| Genus | Brevibacterium | 0.13 | 0.01 | 0.0024 | | | |
| Genus | Ambiguous_taxa | 0.52 | 0.00 | 0.0024 | | | |
| | (Peptostreptococcaceae) | | | | | | |
| Genus | Brachybacterium | 0.10 | 0.01 | 0.0016 | | | |
| Genus | Ruminiclostridium 5 | | | | 1.37 | 0.79 | 0.001 |
| Genus | Candidatus Arthromitus | 1.14 | 0.41 | 0.0023 | | | |
| Genus | [Ruminococcus] torques | | | | 2.27 | 1.72 | 0.063 |
| | group | | | | | | |
| Genus | Ruminiclostridium | 0.06 | 0.02 | 0.0468 | 0.64 | 0.08 | <0.0001 |
| Genus | uncultured bacterium | | | | 6.97 | 2.49 | 0.001 |
| | (Clostridiales vadinBB60 | | | | | | |
| | group) | | | | | | |
| Genus | Ruminococcus 1 | | | | 0.32 | 0.13 | 0.004 |
| Genus | Defluviitaleaceae UCG-011 | | | | 0.13 | 0.03 | 0.001 |
| Genus | Streptococcus | | | | 0.06 | 0.00 | 0.038 |
| Genus | Shuttleworthia | | | | 0.35 | 0.13 | 0.001 |
| Genus | Lachnoclostridium | | | | 1.22 | 0.31 | <0.0001 |
| Genus | Lactobacillus | | | | 13.19 | 20.88 | 0.051 |
| Genus | Lachnospiraceae NK4A136 | | | | 0.53 | 0.07 | <0.0001 |
| | group | | | | | | |
| Genus | Ruminococcaceae UCG-005 | | | | 0.76 | 0.31 | 0.001 |
| Genus | Roseburia | | | | 0.03 | 0.01 | 0.009 |
| Genus | Ruminococcus 2 | | | | 0.02 | 0.02 | 0.016 |
| Genus | Other of Mollicutes RF39 | | | | 0.04 | 0.01 | 0.000 |
| Genus | Harryflintia | | | | 0.04 | 0.01 | <0.0001 |
| Genus | Ruminococcaceae UCG-009 | | | | 0.06 | 0.00 | 0.000 |
| Genus | GCA-900066225 | | | | 0.07 | 0.02 | 0.021 |
| Genus | Family XIII AD3011 group | | | | 0.10 | 0.03 | <0.0001 |
| Genus | Uncultured bacterium of | | | | 0.12 | 0.03 | 0.010 |
| | Mollicutes RF39 | | | | | | |
| Genus | Coprococcus 3 | | | | 0.13 | 0.03 | 0.001 |
| Genus | GCA-900066575 | | | | 0.26 | 0.09 | <0.0001 |
| Genus | Eggerthella | | | | 0.13 | 0.23 | 0.004 |
| Genus | Ruminococcaceae UCG-010 | | | | 0.27 | 0.11 | 0.003 |
| Genus | Christensenellaceae R-7 | | | | 0.35 | 0.20 | 0.029 |
| | group | | | | | | |
| Genus | Erysipelatoclostridium | | | | 0.36 | 0.23 | 0.010 |
| Genus | Ruminococcaceae NK4A214 | | | | 0.59 | 0.11 | 0.001 |
| | group | | | | | | |
| Genus | Negativibacillus | | | | 0.65 | 0.23 | 0.010 |
| Genus | Lachnoclostridium | | | | 1.22 | 0.31 | <0.0001 |
| Genus | Ruminiclostridium 9 | | | | 1.11 | 0.62 | 0.018 |
| Genus | Oscillibacter | | | | 1.26 | 0.57 | 0.009 |
| Genus | Butyricicoccus | | | | 2.21 | 1.38 | 0.003 |
| Genus | Eisenbergiella | | | | 2.83 | 2.12 | 0.027 |
| Genus | Akkermansia | 0.14 | 0.43 | | 0.72 | 4.23 | <0.0001 |
| Genus | uncultured bacterium of | | | | 3.26 | 1.87 | 0.005 |
| | Lachnospiraceae | | | | | | |
| Genus | Faecalibacterium | | | | 5.01 | 1.85 | 0.000 |
| Genus | Ruminococcaceae UCG-014 | | | | 6.91 | 0.92 | <0.0001 |
| Genus | Dietzia | 0.07 | 0.02 | 0.0442 | | | |

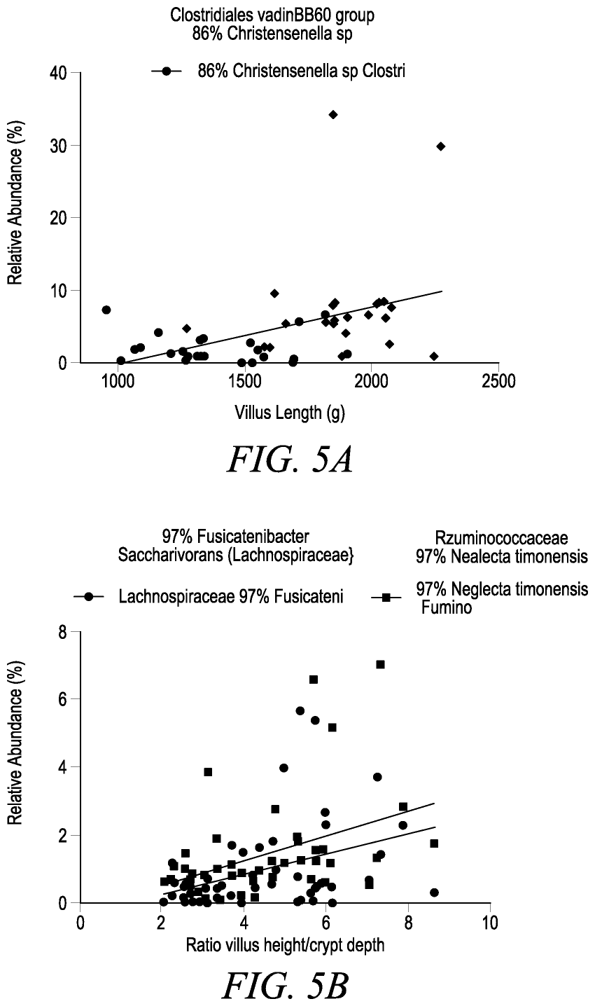

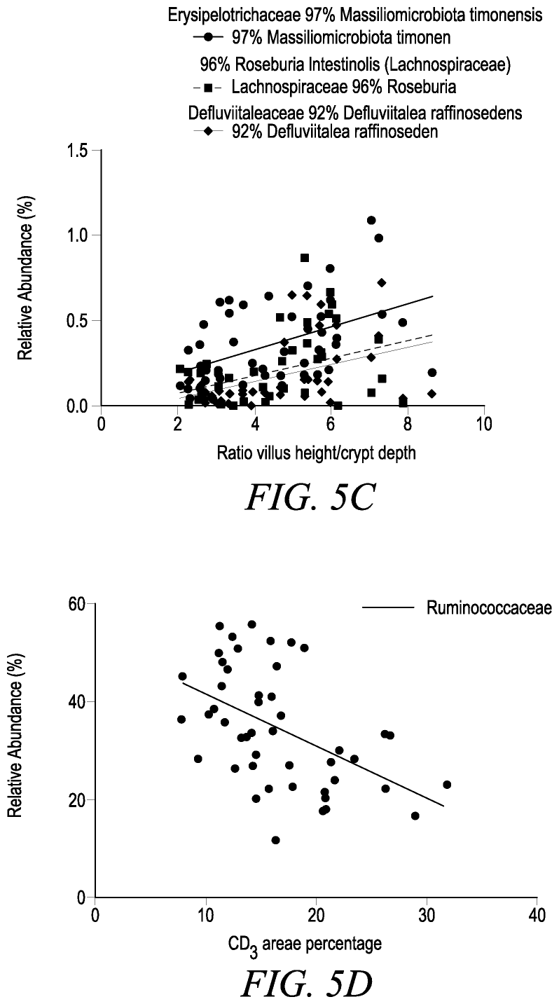

By way of non-limiting example only, histological evaluation of intestinal morphology for selected microorganisms listed in Table 1 confirmed that decreased abundance of the microorganism in challenged chickens correlated with decreased villus length (see FIG. 5A), ratio of villus height to crypt depth (FIG. 5B and FIG. 5C), increased immune cell infiltration (FIG. 5D), and therefore poor intestinal health.

### Example 3

### Identification of Microbial Biomarkers for Intestinal Health in Colon and Caecum Using Modified Diet

A total of 676 day-old broilers (Ross 308) were obtained from a local hatchery and housed in floor pens on wooden shavings. Throughout the study, feed and drinking water were provided ad libitum. The broilers were randomly assigned to two treatment groups, a control and challenge group (13 pens per treatment and 26 broilers per pen). All animals were fed a commercial feed till day 14 and the feed was switched to a wheat based diet supplemented with 20% triticale. From day 14 to 20, all animals from the challenge group received 10 mg florfenicol and 10 mg enrofloxacin per kg body weight via the drinking water daily, to induce substantial changes in the gut microbial community. After the antibiotic treatment, 1 ml of a bacterial cocktail consisting of 10⁸ cfu Escherichia coli (G.78.71), 10⁸ cfu Enterococcus sp. (G.78.62), 10⁸ cfu Lactobacillus salivarius (LMG22873), 10⁷ cfu Lactobacillus crispatus (LMG49479), and 10⁸ cfu Clostridium perfringens (netB-) (D.39.61) was given daily by oral gavage from day 21 till 23. On day 22, the animals were administered a coccidial challenge consisting of 60.000 oocysts of E. acervulina and 30.000 oocysts E. maxima. At day 28, the birds were weighed and 3 birds per pen were euthanized. The duodenal loop was sampled for histological examination and content from caecum and colon was collected for DNA extraction and metabolomics.

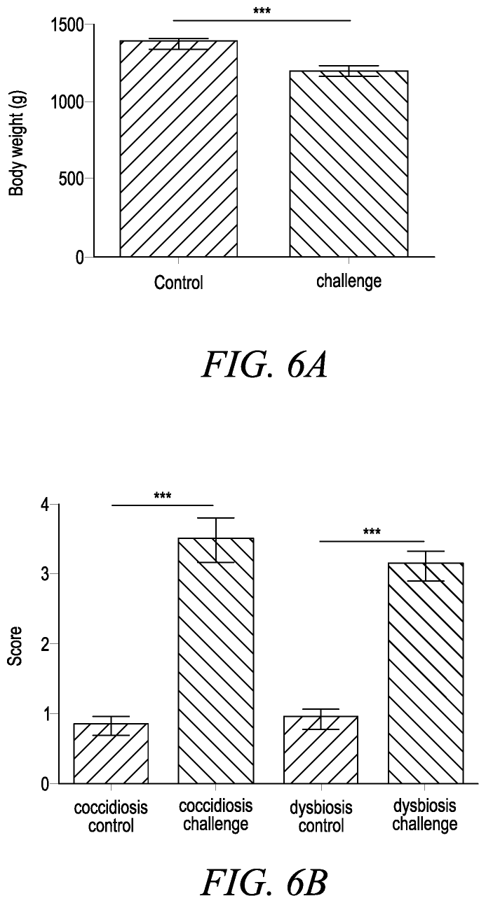

Challenged birds exhibited significant body weight reductions (FIG. 6A) as well as increased dysbiosis and coccidiosis scores (FIG. 6B). Similar to the results displayed in FIG. 2 to FIG. 4 in Example 2, histological evaluation revealed that challenged birds had significantly decreased villus length and increased crypt depth. Decreased villus length and increased crypt depth were both associated with decreased bird body weight. Moreover, challenged birds exhibited significantly increased intestinal immune cell infiltration relative to control animals which was correlated with decreased body weight, increased coccidiosis and dysbiosis score, and villus length.

Statistical analysis of 16S rRNA gene amplicon data was used to identify the taxonomic groups of bacteria in the colonic and caecal microbiota of control and challenged chickens as well as statistically significant changes in their populations following challenge. The results are shown in Table 2.

TABLE 2
Microbiome changes in challenged birds in colon and caecum

| | | Colon | | | Caecum | | |
|---|---|---|---|---|---|---|---|
| Taxa | Bacteria | Control | Challenge | p value | Control | Challenge | p value |
| Order | Rhodospirillales | 0.20% | 0.00% | 0.0276% | | | |
| | Ambiguous_taxa | | | | | | |
| Family | Clostridiales vadinBB60 | | | | 0.11% | 0.08% | 0.0001 |
| | group | | | | | | |
| Family | Peptostreptococcaceae | 0.30% | 0.00% | 0.0001 | | | |
| Genus | Brevibacterium | 0.28% | 0.05% | 0.0594 | | | |
| Genus | Ambiguous_taxa | 0.30% | 0.00% | 0.0001 | | | |
| | (Peptostreptococcaceae) | | | | | | |
| Genus | Brachybacterium | 0.57% | 0.02% | 0.0015 | | | |
| Genus | Ruminiclostridium 5 | 1.01% | 0.50% | 0.0686 | 1.87% | 0.91% | 0.006 |
| Genus | Candidatus Arthromitus | 1.13% | 0.00% | <0.0001 | | | |
| Genus | [Ruminococcus] torques | 1.55% | 3.53% | 0.0059 | | | |
| | group | | | | | | |
| Genus | uncultured bacterium | | | | 0.11% | 0.08% | 0.0001 |
| | (Clostridiales vadinBB60 | | | | | | |
| | group) | | | | | | |
| Genus | Ruminococcus 1 | 0.07% | 0.03% | 0.1294 | | | |
| Genus | Defluviitaleaceae UCG-011 | 0.10% | 0.12% | 0.0293 | 0.23% | 0.09% | 0.0056 |
| Genus | Streptococcus | 0.21% | 0.02% | 0.0012 | | | |
| Genus | Shuttleworthia | 0.20% | 0.17% | 0.0089 | 0.34% | 0.09% | 0.0004 |
| Genus | Lachnoclostridium | 0.77% | 1.26% | 0.0262 | | | |
| Genus | Lactobacillus | 45.14% | 32.71% | 0.0774 | 3.84% | 7.41% | 0.0653 |
| Genus | Lachnospiraceae | | | | 0.49% | 0.20% | 0.0083 |
| | NK4A136 group | | | | | | |
| Genus | Ruminococcaceae UCG-005 | | | | 3.67% | 1.42% | 0.0017 |
| Genus | Helicobacter | 0.03% | 0.00% | 0.0111 | | | |
| Genus | Staphylococcus | 0.04% | 0.01% | 0.0952 | | | |
| Genus | uncultured Firmicutes | 0.02% | 0.03% | 0.0546 | | | |
| | bacterium | | | | | | |
| Genus | Jeotgalicoccus | 0.07% | 0.01% | 0.0042 | | | |
| Genus | Anaerofilum | 0.02% | 0.10% | 0.0206 | | | |
| Genus | Marvinbryantia | 0.17% | 0.06% | 0.0807 | 0.13% | 0.06% | |
| Genus | Ruminococcaceae UCG-013 | 0.25% | 0.08% | 0.0088 | | | |
| Genus | Intestinimonas | 0.12% | 0.24% | 0.1715 | | | |
| Genus | Enterococcus | 0.28% | 0.15% | 0.0803 | | | |
| Genus | Fournierella | 0.05% | 0.42% | 0.6307 | | | |
| Genus | UC5-1-2E3 | 0.18% | 0.41% | 0.4252 | | | |
| Genus | Barnesiella | 0.29% | 0.37% | 0.2099 | | | |
| Genus | Sellimonas | 0.28% | 0.59% | 0.0173 | 0.34% | 0.72% | 0.0456 |
| Genus | Corynebacterium 1 | 0.85% | 0.24% | 0.0223 | | | |
| Genus | Bifidobacterium | 0.07% | 1.67% | <0.0001 | 0.49% | 1.51% | 0.0002 |
| Genus | Tyzzerella | 0.55% | 1.86% | 0.0017 | | | |
| Genus | Clostridium sensu stricto 1 | 0.11% | 2.71% | 0.0297 | 0.01% | 0.41% | 0.0358 |
| Genus | Escherichia-Shigella | 1.31% | 3.72% | 0.0256 | 1.31% | 3.74% | 0.0238 |
| Genus | Subdoligranulum | 4.64% | 2.34% | 0.3412 | | | |
| Genus | Bacteroides | 4.28% | 16% | 0.0004 | | | |
| Genus | Lachnospiraceae ASF356 | | | | 0.15% | 0.02% | 0.005 |
| Genus | Lachnospiraceae UC5-1-2E3 | | | | 0.17% | 0.42% | 0.8536 |

### Example 4

### Identification of Metabolic Biomarkers Correlated with Intestinal Health

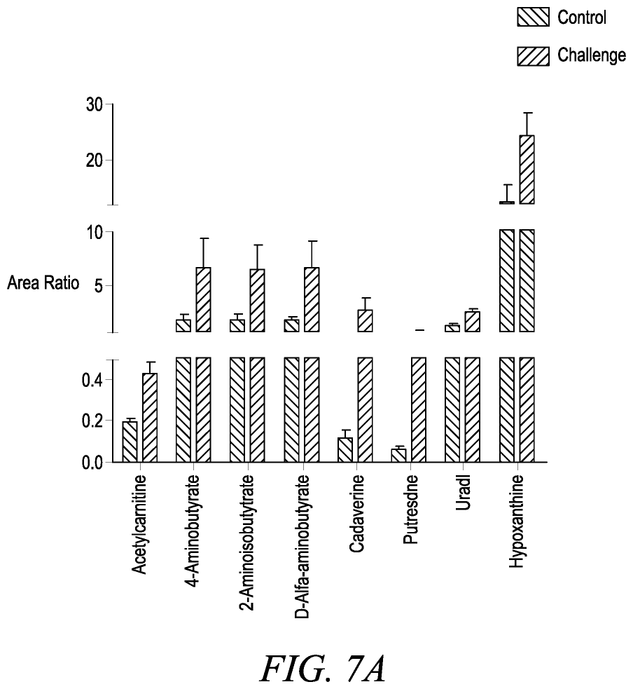

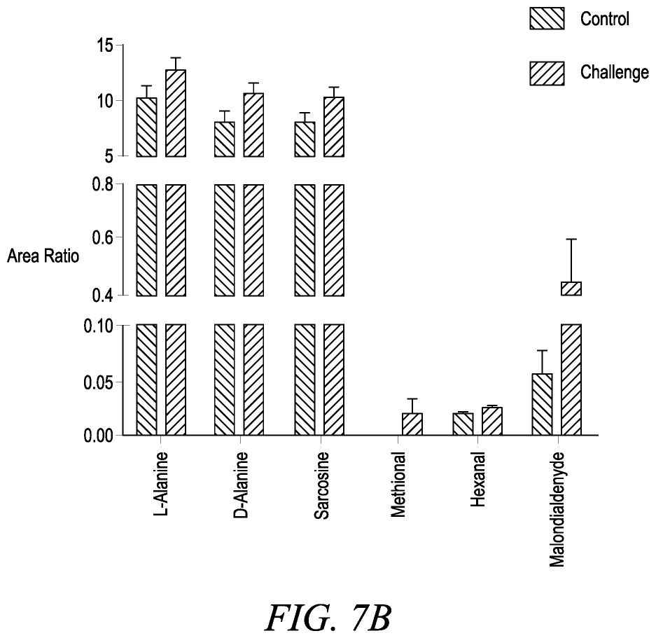

A further metabolomic analysis of colon and caecum samples derived from the control and challenged animals of Example 3 was performed. As shown in FIG. 7A and FIG. 7B, a number of metabolites were observed in both the colon (FIG. 7A) and caecum (FIG. 7B) of challenged chickens at levels significantly higher in comparison to their corresponding levels in control chickens. In addition to the metabolites shown in FIG. 7A and FIG. 7B, the following additional compounds were found in the intestines of challenged chickens at levels significantly higher than those found in unchallenged controls: linoleyl carnitine, linalool, 3-[(9Z)-9-octadecenoyloxy]-4-(trimethylammonio) butanoate, (−)-trans-methyl dihydrojasmonate, icomucret, 1,3-dioctanoylglycerol, and ethyl 2-nonynoate. Thus, the presence of one or more of these compounds at levels significantly higher than healthy control animals is correlated with poor intestinal health and their presence and quantification can be used to assess and predict the intestinal health of poultry.

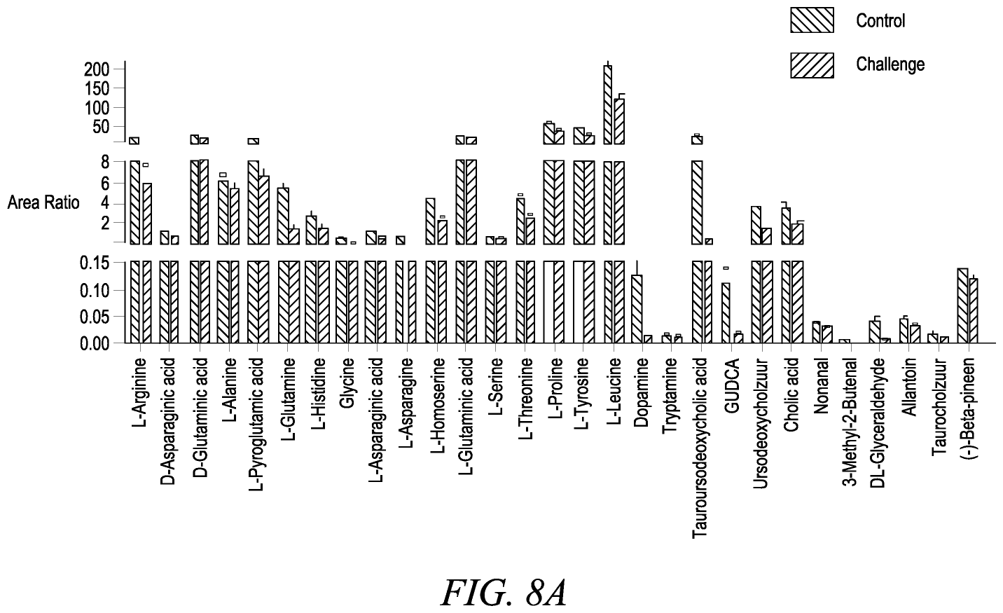

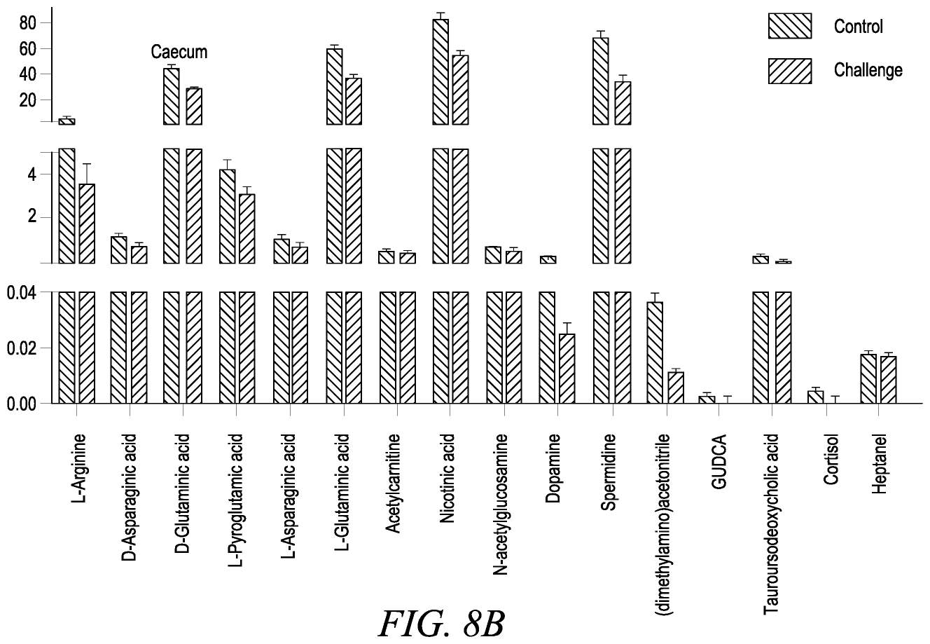

As shown in FIG. 8A and FIG. 8B, additional metabolites were identified in both the colon (FIG. 8A) and caecum (FIG. 8B) of challenged chickens at levels significantly lower in comparison to their corresponding levels in control chickens (i.e., these compounds were present at statistically significant higher levels in healthy unchallenged animals). In addition to the metabolites shown in FIG. 8A and FIG. 8B, the following additional compounds were found in the intestines of challenged chickens at levels significantly lower than those found in unchallenged controls (i.e., these compounds are more present in healthy unchallenged control animals): 5-(2-carboxyethyl)-2-hydroxyphenyl beta-D-glucopyranosiduronic acid, 4,15-Diacetoxy-3-hydroxy-12,13-epoxytrichothec-9-en-8-yl3-hydroxy-3-methylbutanoate, scoparone, asp-leu, ethyl benzoylacetate, L-(+)-glutamine, 1-allyl-2,3,4,5-tetramethoxybenzene, (DL)-3-O-methyldopa, dictyoquinazol A, 1-(3-furyl)-7-hydroxy-4,8-dimethyl-1,6-nonanedione methyl 3,4,5-trimethoxycinnamate, and butylparaben. Thus, the presence of one or more of these compounds at levels significantly lower than healthy control animals is correlated with poor intestinal health and their presence and quantification can be used to assess and predict the intestinal health of poultry.

### Example 5

### Verification of Microbial Biomarkers for Intestinal Health in Working European Varms

At 6 farms located in Flanders, Belgium, 10 broilers aging 27-28 days, were weighted and euthanized to collect colon and caecal content. At 4 other farms in Flanders, 10 broilers aging 28 days were weighted, euthanized and only colon content was sampled. From each intestinal sample, 100 mg was weighted and used for DNA extraction according to the protocol described in previous examples. DNA was used for the library preparation as described in previous examples and sequencing according to previous examples. From each bird the duodenal loop was sampled and processed according previous examples. Correlations were calculated using the relative abundance of all families and genera versus each bird parameter being, body weight, CD3 area percentage and ratio between villus length and crypt depth.

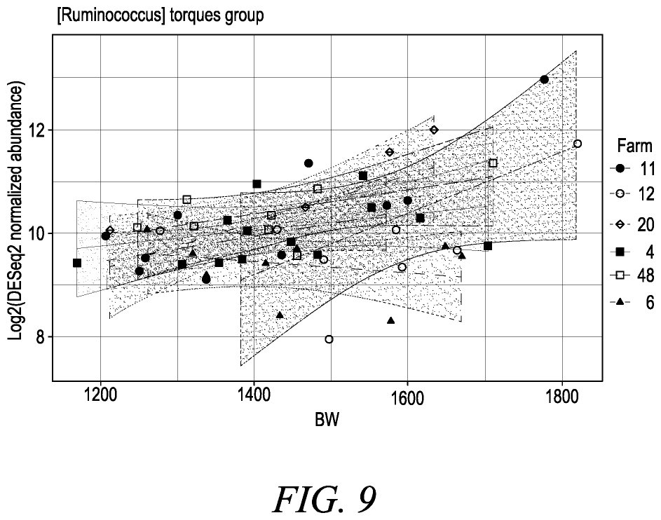

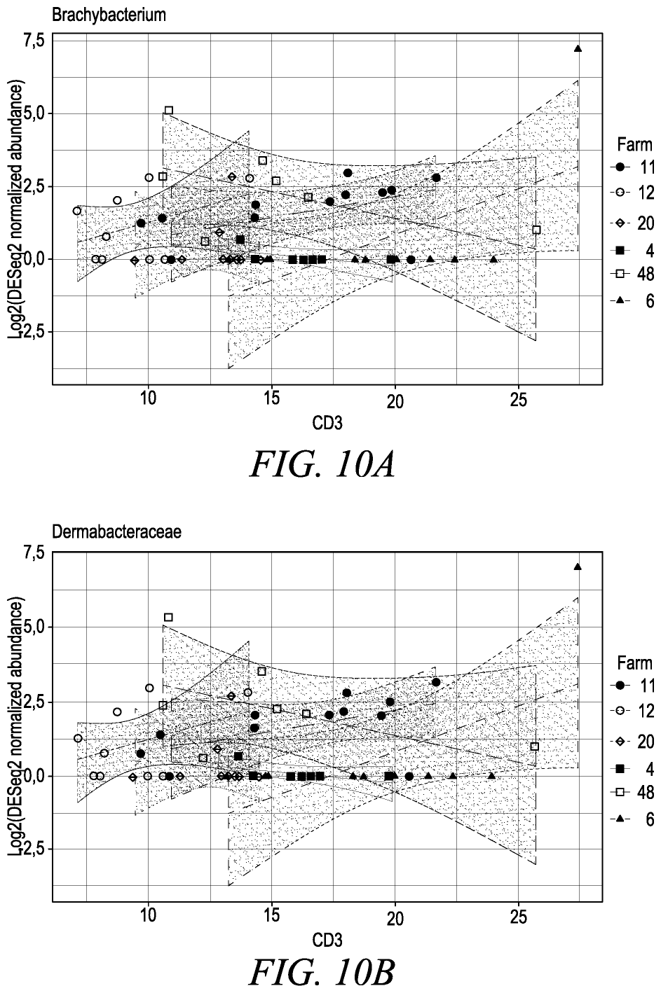

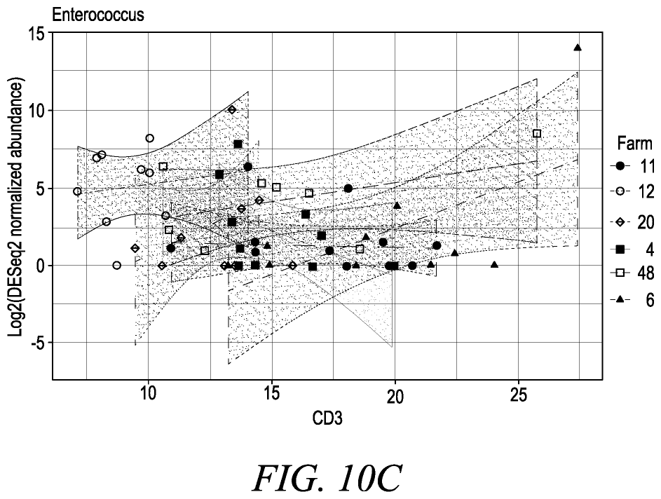

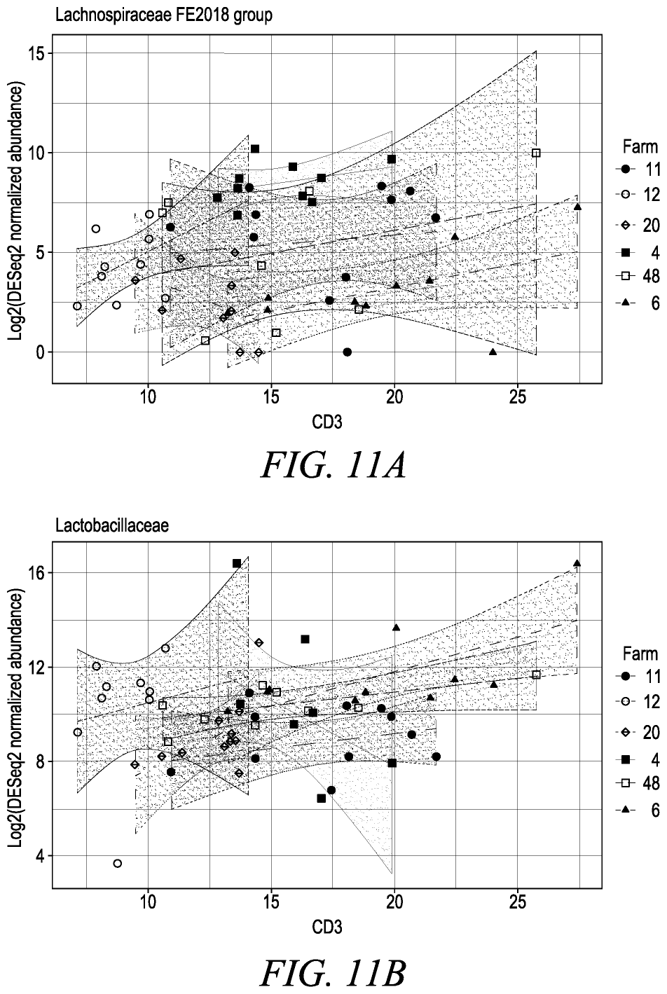

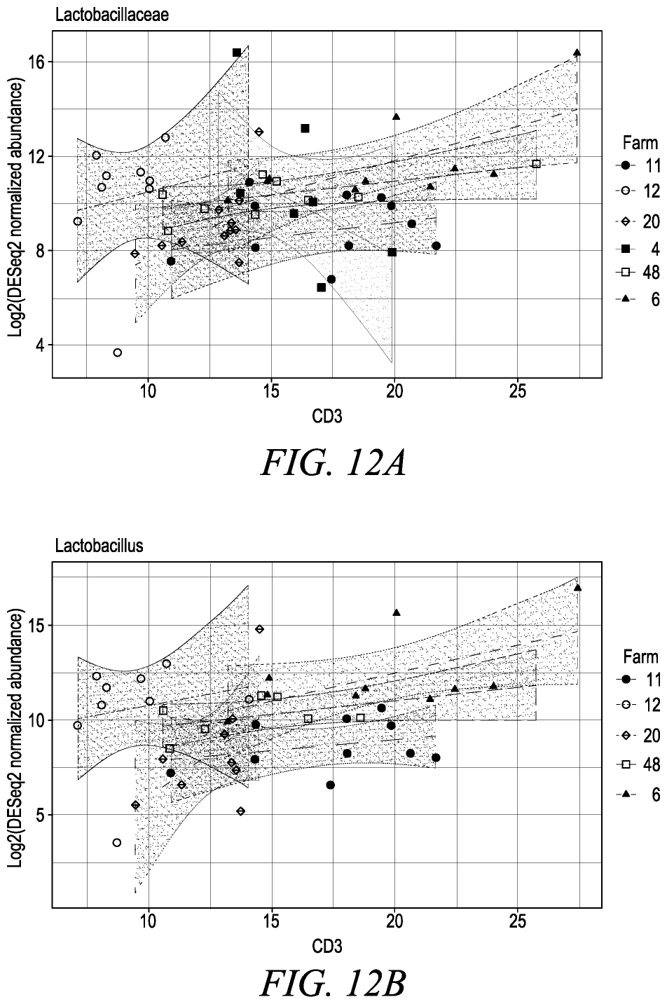

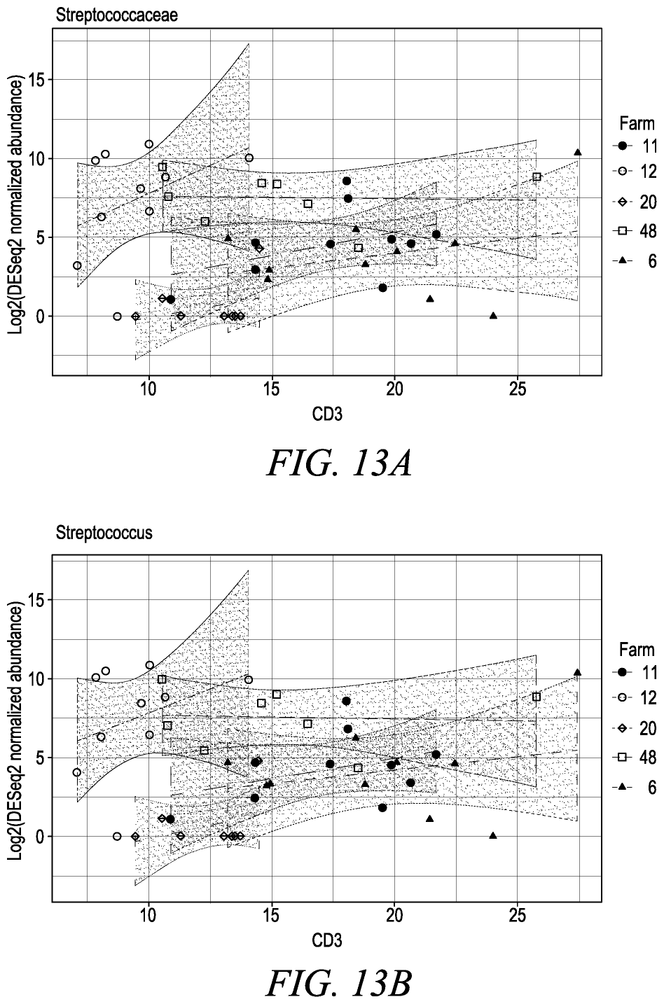

As shown in FIG. 9, At the majority of the farms there is a positive correlation between Ruminococcus torques group in the caecum and the body weight. Multiple bacterial populations present in the caecum at the majority of farms showed a positive correlation with the CD3 area percentage. These included Brachybacterium (FIG. 10A), Dermabacteraceae (FIG. 10B), and Enterococcus (FIG. 10C). Moreover, at the majority of the farms there was a positive correlation between bacteria in the caecum belonging to the family Lachnospiraceae and the CD3 area percentage (FIG. 11A). As shown in FIG. 11B, the Lachnospiraceae FE2018 group seems to be responsible for the correlation between the family Lachnospiraceae and the CD3 area percentage. Similarly, at the majority of the farms there is a positive correlation between bacteria in the caecum belonging to the family Lactobacillaceae and the CD3 area percentage (FIG. 12A). As shown in FIG. 12B, Lactobacillus seems to be responsible for the correlation between the family Lactobacillaceae and the CD3 area percentage. Bacteria in the caecum belonging to the family Streptococcaceae show positive correlation with the CD3 area percentage (FIG. 13A). As shown in FIG. 13B, Streptococcus seems to be responsible for the correlation between the family Streptococcaceae and the CD3 area percentage.

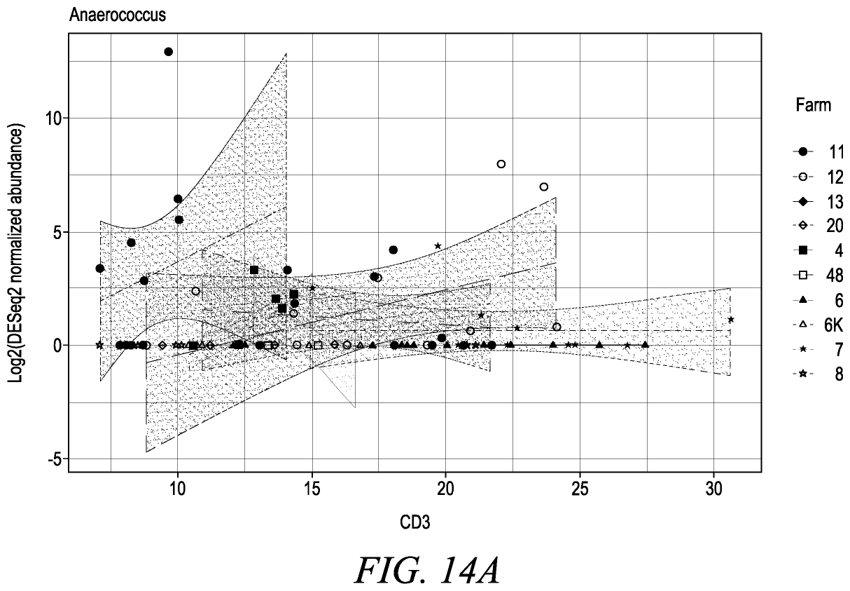

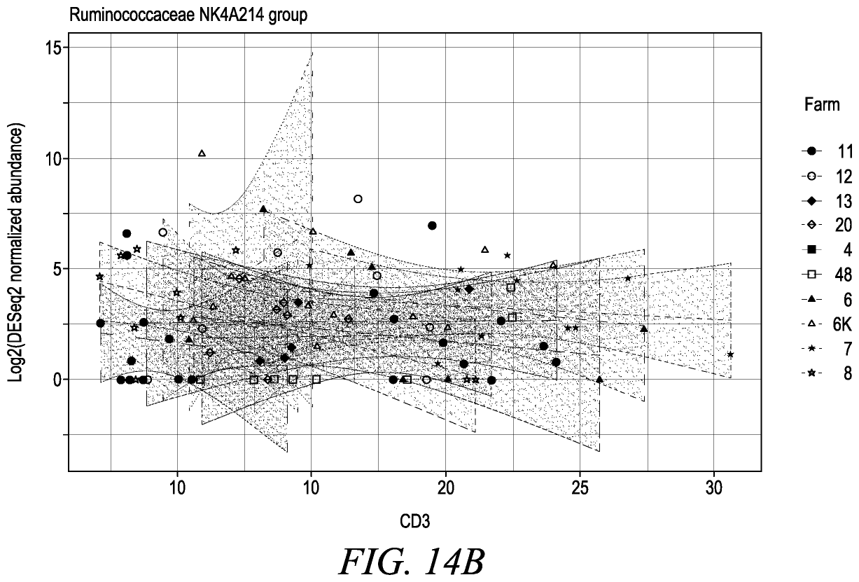

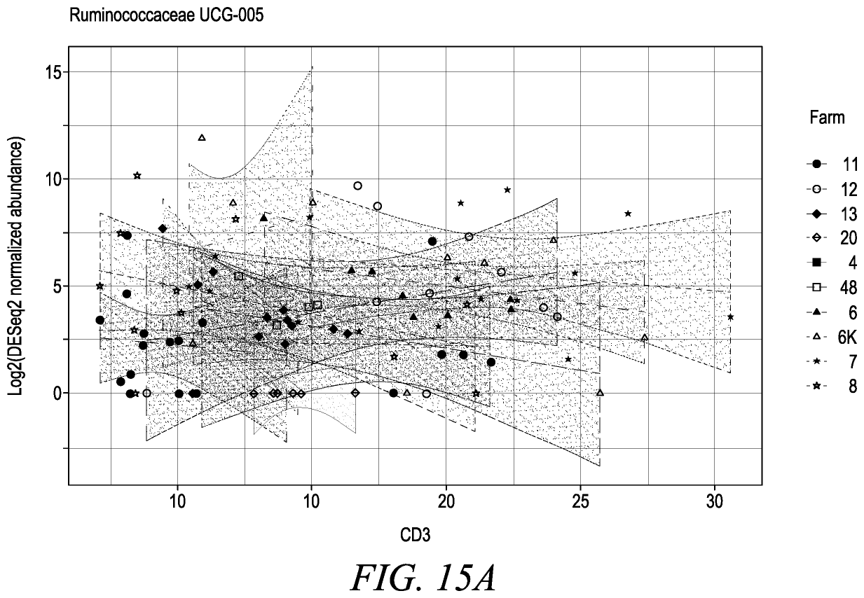

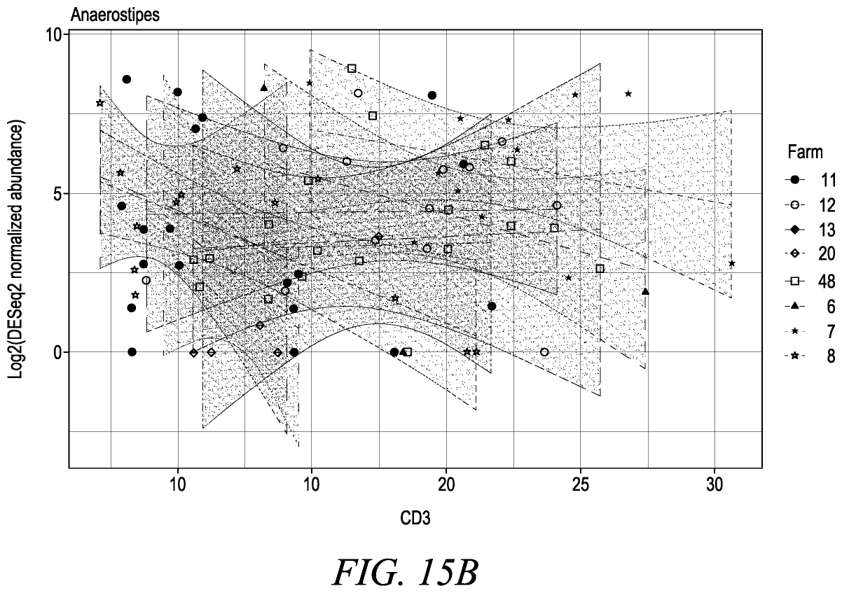

In the colon, several bacterial populations showed a correlation with the concentration of infiltrated immune cells in the duodenum (CD3 area percentage). As shown in FIG. 14A, at the majority of the farms where Anaerococcus bacteria are present in the colon there is a positive correlation with the CD3 area percentage. Conversely, at the majority of the farms where Ruminococcaceae NK4A214 bacteria are present in the colon there is a negative correlation with the CD3 area percentage (FIG. 14B). At the majority of the farms where Ruminococcaceae UCG-005 bacteria are present in the colon there is a negative correlation with the CD3 area percentage (FIG. 15A). Also, a negative correlation between Anaerostipes (from the family of Lachnospiraceae) in the colon and the CD3 area percentage was observed at the majority of farms (FIG. 15B). Moreover, a negative correlation between Lachnoclostridium (FIG. 16A), Ruminiclostridium 5 (FIG. 16B), and Ruminiclostridium 9 (FIG. 16C) in the colon was observed at a majority of farms.

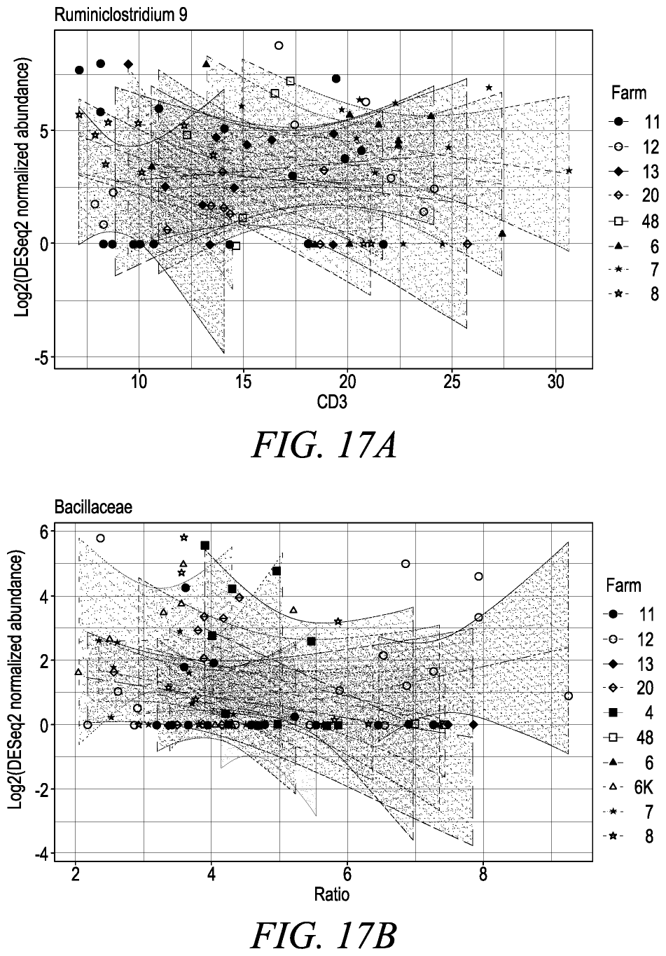

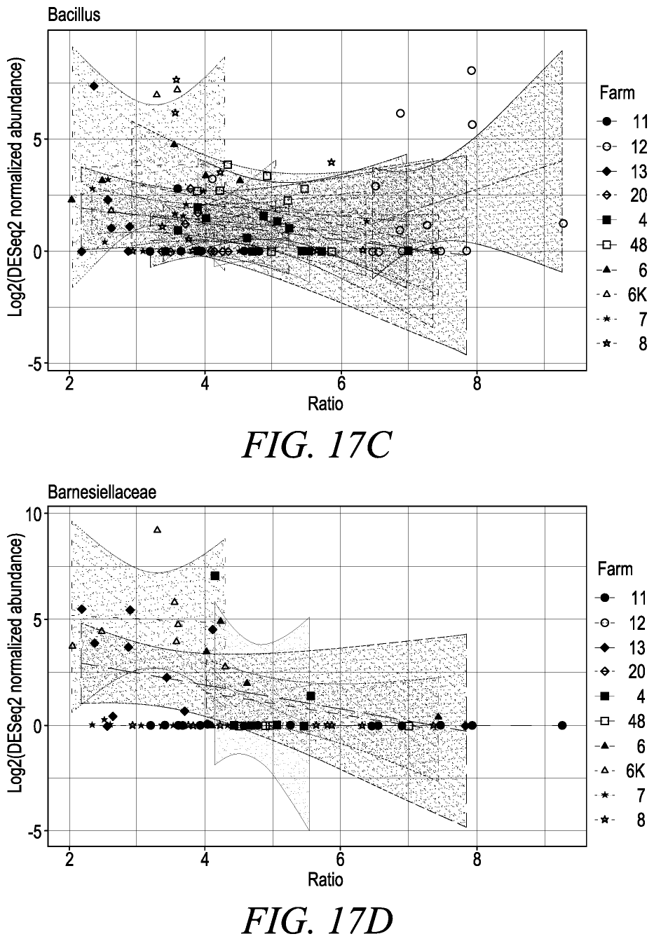

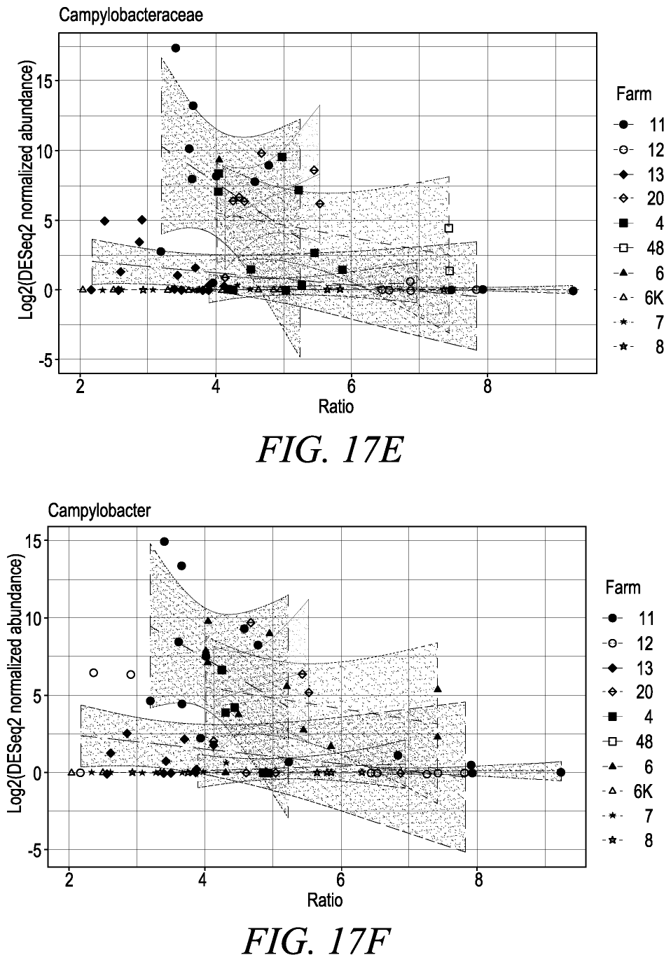

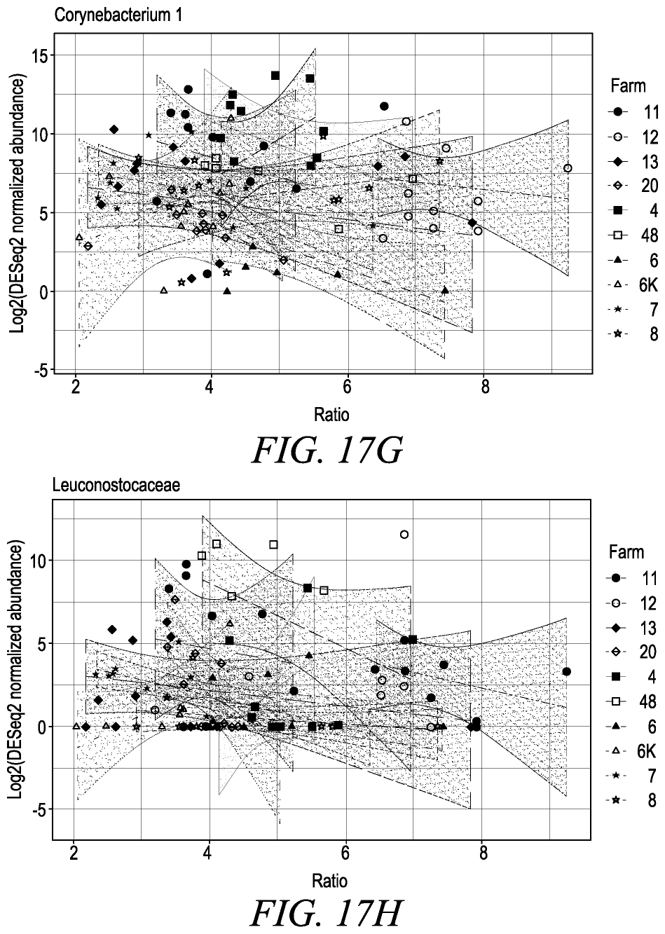

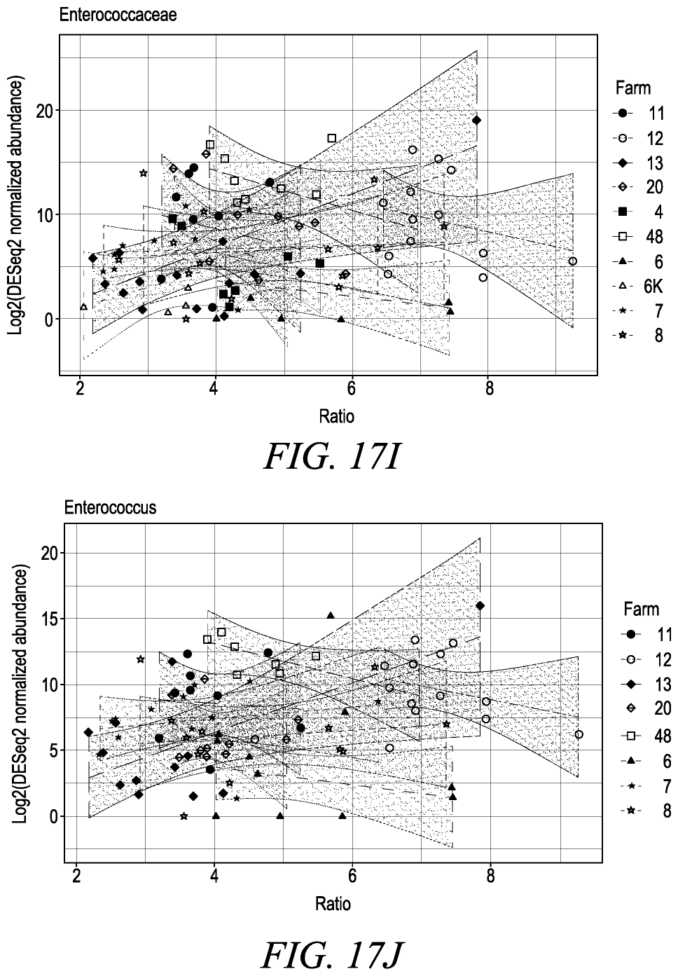

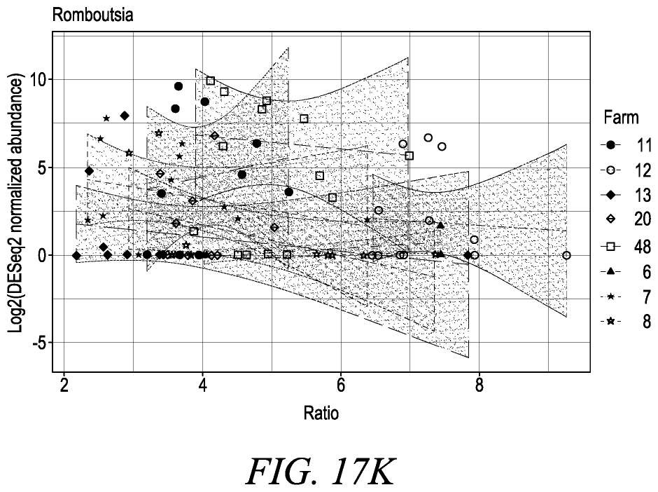

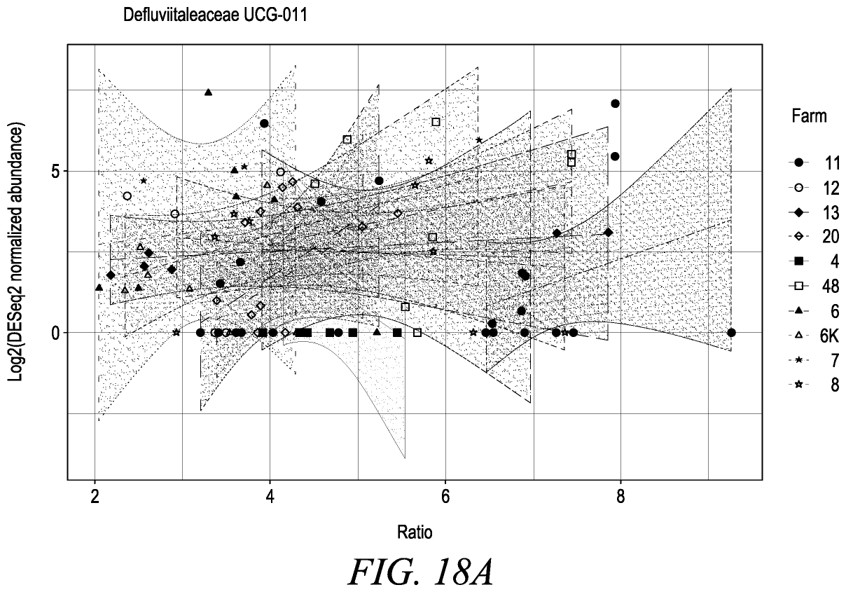

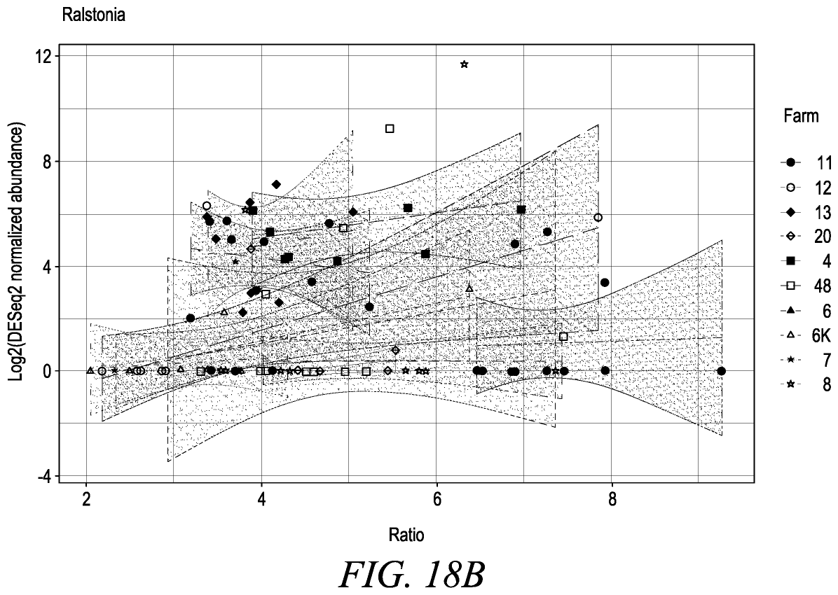

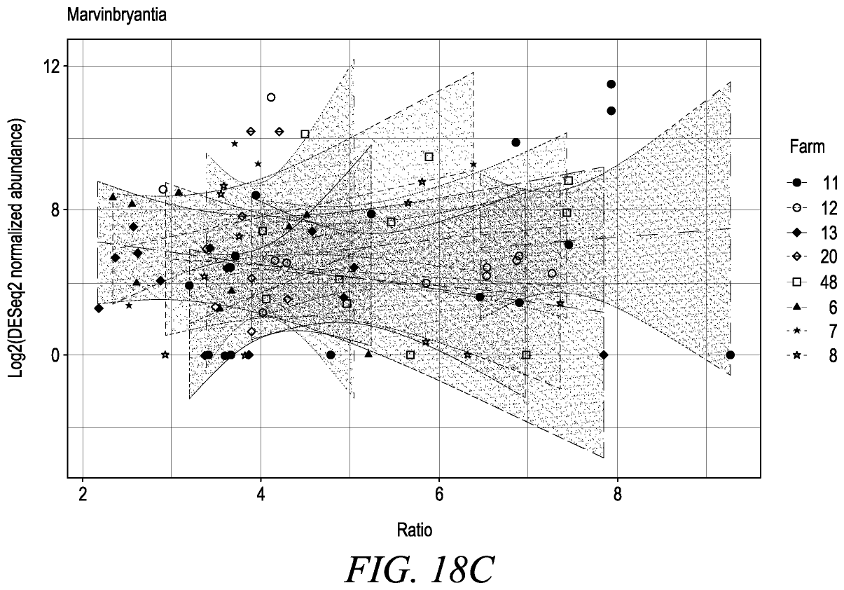

Additionally, bacterial populations in the colon showed a negative correlation with the ratio between villus length and crypt depth (“the ratio”). For example, at the majority of the farms where Anaerococcus (FIG. 17A), Bacillaceae (FIG. 17B), Barnesiellaceae (FIG. 17D), Campylobacteraceae (FIG. 17E), Corynebacterium 1 (FIG. 17G), Leuconostocaceae (FIG. 1711), Enterococcaceae (FIG. 17I), Romboutsia (FIG. 17K) was present in the colon there is a negative correlation with the ratio between villus length and crypt depth. For Bacillaceae, Bacillus seems to be responsible for the correlation between the family Bacillaceae and the ratio (FIG. 17C). For Campylobacteraceae, Campylobacter seems to be responsible for the correlation between the family Campylobacteraceae and the ratio (FIG. 17F). For Enterococcaceae, Enterococcus seems to be responsible for the correlation between the family Enterococcaceae and the ratio (FIG. 17J).

Conversely, a positive correlation with the ratio between villus length and crypt depth was observed at the majority of farms where Defluviitaleaceae UCG-011 (FIG. 18A), Ralstonia (FIG. 18B) and Marvinbryantia (FIG. 18C) was present in the colon.

We claim:

1. A method for determining the intestinal health status of a domesticated bird comprising:
quantifying populations of one or more microorganism(s) in a fecal and/or intestinal content sample from the bird selected from the group consisting of: a microorganism from the Clostridiales vadinBB60 group family of microorganisms and a microorganism from the Peptostreptococcaceae family of microorganisms,
wherein a decreased population of said one or more microorganism(s) in said fecal or intestinal content sample, when compared to the level found in fecal or intestinal content samples of healthy control animals, is an indicator of poor intestinal health.

2. The method of claim 1, further comprising quantifying populations of one or more microorganism(s) in a fecal and/or intestinal content sample from the bird selected from the group consisting of: a microorganism from the genus Brevibacterium, Brachybacterium, Ruminiclostridium, Candidatus Arthromitus, Ruminococcus optionally with the exception of Ruminococcus torques, Streptococcus, Shuttleworthia, Lachnospiraceae NK4A136 group, and Ruminococcaceae UCG-005,
wherein a decreased population of said one or more microorganism(s) in said fecal or intestinal content sample, when compared to the level found in fecal or intestinal content samples of healthy control animals, is an indicator of poor intestinal health.

3. The method of claim 1 or claim 2, wherein the intestinal content sample is obtained from ileum, colon, or caecum.

4. The method of any one of claims 1-3, further comprising quantifying populations of one or more microorganism(s) in an intestinal content sample from the bird selected from: a microorganism from the genus Defluviitaleaceae UCG-011, a microorganism from the genus Lachnoclostridium, or a microorganism from the Ruminococcus torques group,
(a) wherein a decreased population of said one or more microorganism(s) obtained from the caecum, when compared to the level found in caecum samples of healthy control animals, is an indicator of poor intestinal health; and/or
(b) wherein an increased population of said one or more microorganism(s) obtained from the colon, when compared to the level found in colon samples of healthy control animals, is an indicator of poor intestinal health.

5. The method of any one of claims 1-4, further comprising quantifying populations of one or more microorganism(s) in an intestinal content sample from the bird a microorganism from the genus Lactobacillus,
(a) wherein an increased population of said one or more microorganism(s) obtained from the caecum, when compared to the level found in caecum samples of healthy control animals, is an indicator of poor intestinal health; and/or
(b) wherein a decreased population of said one or more microorganism(s) obtained from the colon, when compared to the level found in colon samples of healthy control animals, is an indicator of poor intestinal health.

6. The method of any one of claims 1-5, further comprising quantifying populations of one or more microorganism(s) in a fecal and/or intestinal content sample from the bird selected from
(a) a microorganism from the phylum Tenericutes and/or Firmicutes;
(b) a microorganism from the phylum Verrucomicrobia and/or Bacteroidetes;
(c) a microorganism from the class Mollicutes RF39, Erysipelotrichales, Clostridiales, and/or Micrococcales;
(d) a microorganism from the class Coriobacteriales, Verrucomicrobiales, and/or Bacteroidales
(e) a microorganism from the family Streptococcaceae, Defluviitaleaceae, Christensenellaceae, Erysipelotrichaceae, Lachnospiraceae, Ruminococcaceae, Dermabacteraceae, Brevibacteriaceae, and/or Dietziaceae;
(f) a microorganism from the family Eggerthellaceae, Akkermansiaceae, Lactobacillaceae, and/or Clostridiaceae;
(g) a microorganism from the genus Roseburia, Harryflintia, Ruminococcaceae UCG-009, Coprococcus, Ruminococcaceae UCG-010, Ruminococcus, Christensenellaceae R-7 group, Erysipelatoclostridium, Ruminococcaceae NK4A214 group, Negativibacillus, Oscillibacter, Butyricicoccus, and/or Eisenbergiella; and/or
(h) a microorganism from the genus Eggerthella, and/or Akkermansia,
(1) wherein a decreased population of said one or more microorganism(s) from (a), (c), (e), and/or (h) in said fecal or intestinal content sample, when compared to the level found in fecal or intestinal content samples of healthy control animals, is an indicator of poor intestinal health; and/or
(2) wherein an increased population of said one or more microorganism(s) from (b), (d), (f), and/or (g) in said fecal or intestinal content sample, when compared to the level found in fecal or intestinal content samples of healthy control animals, is an indicator of poor intestinal health.

7. The method of claim 6, wherein the intestinal content sample is obtained from ileum and/or caecum.

8. The method of any one of claims 1-5, further comprising quantifying populations of one or more microorganism(s) in a fecal and/or intestinal content sample from the bird selected from
(a) a microorganism from the order Rhodospirillales;
(b) a microorganism from the genus Helicobacter, Staphylococcus, Jeotgalicoccus, Ruminococcus, Marvinbryantia, Ruminococcaceae UCG-013, Enterococcus, Corynebacterium, and/or Subdoligranulum; and/or
(c) a microorganism from the genus Firmicutes, Anaerofilum, Intestinimonas, Fournierella, Barnesiella, Barnesiella, Bifidobacterium, Tyzzerella, Clostridium sensu stricto, and/or Escherichia-Shigella,
(1) wherein a decreased population of said one or more microorganism(s) from (a) and/or (b) in said fecal or intestinal content sample, when compared to the level found in fecal or intestinal content samples of healthy control animals, is an indicator of poor intestinal health; and/or
(2) wherein an increased population of said one or more microorganism(s) from (c) in said fecal or intestinal content sample, when compared to the level found in fecal or intestinal content samples of healthy control animals, is an indicator of poor intestinal health.

9. The method of claim 8, wherein the intestinal content sample is obtained from colon and/or caecum.

10. The method of any one of claims 1-9, wherein intestinal health is determined by one or more of (a) measuring villus length in the duodenum of the birds; (b) measuring villus-to crypt ratio in the duodenum of the birds; (c) measuring T-lymphocyte infiltration in villi; and/or (d) scoring the macroscopic gut appearance of the birds.

11. The method of any one of claims 1-10, wherein the domesticated bird is selected from the group consisting of chickens, turkeys, ducks, geese, emus, ostriches, quail, and pheasant.

12. The method of claim 11, wherein the chicken is a broiler.

13. The method of any one of claims 1-12, wherein said one or more microorganism(s) are quantified by using antibodies which specifically bind to said microorganism.

14. The method of claim 13, wherein said antibodies are part of an Enzyme-Linked Immuno Sorbent Assay (ELISA).

15. The method of any one of claims 1-14, wherein said one or more microorganisms are identified and quantified by real-time PCR.

16. The method of claim 15, further comprising sequencing the 16S ribosomal DNA (rDNA) gene.

17. The method of any one of claims 1-16, further comprising quantifying one or more metabolite(s) in a fecal and/or intestinal content sample from the bird selected from the group consisting of linoleyl carnitine, linalool, 3-[(9Z)-9-octadecenoyloxy]-4-(trimethylammonio)butanoate, (−)-trans-methyl dihydrojasmonate, icomucret, 1,3-dioctanoylglycerol, ethyl 2-nonynoate, 4-aminobutyrate, 2-amino-isobutyrate, D-alpha-aminobutyrate, cadaverine, putrescine, uracil, hypoxanthine, D-alanine, sarcosine, methional, hexanal, malondialdehyde, L-alanine, and acetylcarnitine,
wherein an increased level of said one or more metabolite(s) in said fecal or intestinal content sample, when compared to the level found in fecal or intestinal content samples of healthy control animals, is an indicator of poor intestinal health.

18. The method of any one of claims 1-17, further comprising quantifying one or more metabolite(s) in a fecal and/or intestinal content sample from the bird selected from the group consisting of 5-(2-carboxyethyl)-2-hydroxyphenyl beta-D-glucopyranosiduronic acid, 4,15-Diacetoxy-3-hydroxy-12,13-epoxytrichothec-9-en-8-yl3-hydroxy-3-methylbutanoate, scoparone, asp-leu, ethyl benzoylacetate, L-(+)-glutamine, 1-allyl-2,3,4,5-tetramethoxybenzene, (DL)-3-O-methyldopa, dictyoquinazol A, 1-(3-furyl)-7-hydroxy-4,8-dimethyl-1,6-nonanedione methyl 3,4,5-trimethoxycinnamate, butylparaben, aspartic acid, L-arginine, glutamic acid, L-pyroglutamic acid, L-glutamine, L-histidine, glycine, (−)-beta-pineen, L-asparagine, L-homoserine, L-serine, L-threonine, L-prdine, L-tyrosine, L-leucine, dopamine, taurocholic acid, tryptamine, tauroursodeoxychdic acid, glycoursodeoxycholic acid, ursodeoxycholic acid, cholic acid, nonanal, 3-methyl-2-butenal, DL-glyceraldehyde, allantoin, nicotinic acid, N-acetylglucosamine, spermidine, (dimethylamino)acetonitrile, glycoursodeoxycholic acid, tauroursodeoxycholic acid, cortisol, and heptanal,
wherein a decreased level of said one or more metabolite(s) in said fecal or intestinal content sample, when compared to the level found in fecal or intestinal content samples of healthy control animals, is an indicator of poor intestinal health.

19. The method of claim 17 or claim 18, wherein said one or more metabolite(s) are quantified by using antibodies which specifically bind to said metabolite.

20. The method of claim 19, wherein said antibodies are part of an Enzyme-Linked Immuno Sorbent Assay (ELISA).

21. The method of claim 17 or claim 18, wherein said one or more metabolite(s) are quantified by using mass spec or HPLC.

22. A method for quantifying one or more microorganism(s) from a domesticated bird at risk for or thought to be at risk for poor intestinal health comprising: quantifying one or more microorganism(s) in a sample selected from the group consisting of a microorganism from the Clostridiales vadinBB60 group family of microorganisms and a microorganism from the Pepto streptococcaceae family of microorganisms, wherein the sample is a fecal or an intestinal content sample.

23. The method of claim 22, further comprising quantifying populations of one or more microorganism(s) in the sample from the bird selected from the group consisting of: Brevibacterium, Brachybacterium, Ruminiclostridium, Candidatus Arthromitus, Ruminococcus with the optional exception of Ruminococcus torques, Streptococcus, Shuttleworthia, Lachnospiraceae NK4A136 group, and Ruminococcaceae UCG-005.

24. The method of claim 22 or claim 23, wherein the intestinal content sample is obtained from ileum, colon, or caecum.

25. The method of any one of claims 22-24, further comprising quantifying populations of one or more microorganism(s) in an intestinal content sample from the bird selected from: a microorganism from the genus Defluviitaleaceae UCG-011, a microorganism from the genus Lachnoclostridium, a microorganism from the genus Lactobacillus, or a microorganism from the Ruminococcus torques group, wherein the intestinal content sample is obtained from colon or caecum.

26. The method of any one of claims 22-25, further comprising quantifying populations of one or more microorganism(s) in a fecal and/or intestinal content sample from the bird selected from
(a) a microorganism from the phylum Tenericutes, Verrucomicrobia, Bacteroidetes, and/or Firmicutes;
(b) a microorganism from the class Mollicutes RF39, Erysipelotrichales, Clostridiales, Coriobacteriales, Verrucomicrobiales, Bacteroidales, and/or Micrococcales;
(c) a microorganism from the order Rhodospirillales;
(d) a microorganism from the family Streptococcaceae, Defluviitaleaceae, Christensenellaceae, Erysipelotrichaceae, Lachnospiraceae, Ruminococcaceae, Dermabacteraceae, Brevibacteriaceae, Dietziaceae, Eggerthellaceae, Akkermansiaceae, Lactobacillaceae, and/or Clostridiaceae; and/or
(e) a microorganism from the genus Roseburia, Harryflintia, Ruminococcaceae UCG-009, Coprococcus, Ruminococcaceae UCG-010, Ruminococcus, Christensenellaceae R-7 group, Erysipelatoclostridium, Ruminococcaceae NK4A214 group, Negativibacillus, Oscillibacter, Butyricicoccus, Eggerthella, Akkermansia, Helicobacter, Staphylococcus, Jeotgalicoccus, Ruminococcus, Marvinbryantia, Ruminococcaceae UCG-013, Enterococcus, Corynebacterium, Subdoligranulum, Firmicutes, Anaerofilum, Intestinimonas, Fournierella, Barnesiella, Barnesiella, Bifidobacterium, Tyzzerella, Clostridium sensu stricto, Escherichia-Shigella, and/or Eisenbergiella;
wherein the intestinal content sample is obtained from colon and/or caecum.

27. The method of any one of claims 22-26, wherein the domesticated bird is selected from the group consisting of chickens, turkeys, ducks, geese, quail, and pheasant.

28. The method of claim 27, wherein the chicken is a broiler.

29. The method of any one of claims 22-28, wherein said one or more microorganism(s) are quantified by using antibodies which specifically bind to said microorganism.

30. The method of claim 29, wherein said antibodies are part of an Enzyme-Linked Immuno Sorbent Assay (ELISA).

31. The method of any one of claims 22-28, wherein said one or more microorganisms are identified and quantified by real-time PCR.

32. The method of claim 31, further comprising sequencing the 16S ribosomal DNA (rDNA) gene.

33. The method of any one of claims 22-32, further comprising (a) measuring villus length in the duodenum of the birds; (b) measuring villus-to crypt ratio in the duodenum of the birds; (c) measuring T-lymphocyte infiltration in villi; and/or (d) scoring the macroscopic gut appearance of the birds.

34. The method of any one of claims 22-33, further comprising quantifying one or more metabolite(s) in a fecal and/or intestinal content sample from the bird selected from the group consisting of linoleyl carnitine, linalool, 3-[(9Z)-9-octadecenoyloxy]-4-(trimethylammonio)butanoate, (−)-trans-methyl dihydrojasmonate, icomucret, 1,3-dioctanoylglycerol, ethyl 2-nonynoate, 4-aminobutyrate, 2-amino-isobutyrate, D-alpha-aminobutyrate, cadaverine, putrescine, uracil, hypoxanthine, D-alanine, sarcosine, methional, hexanal, malondialdehyde L-alanine, and acetylcarnitine, wherein the sample is a fecal or an intestinal content sample.

35. The method of any one of claims 22-34, further comprising quantifying one or more metabolite(s) in a fecal and/or intestinal content sample from the bird selected from the group consisting of 5-(2-carboxyethyl)-2-hydroxyphenyl beta-D-glucopyranosiduronic acid, 4,15-Diacetoxy-3-hydroxy-12,13-epoxytrichothec-9-en-8-yl 3-hydroxy-3-methylbutanoate, scoparone, asp-leu, ethyl benzoylacetate, L-(+)-glutamine, 1-allyl-2,3,4,5-tetramethoxybenzene, (DL)-3-O-methyldopa, dictyoquinazol A, 1-(3-furyl)-7-hydroxy-4,8-dimethyl-1,6-nonanedione methyl 3,4,5-trimethoxycinnamate, butylparaben, aspartic acid, L-arginine, glutamic acid, L-pyroglutamic acid, L-glutamine, L-histidine, glycine, (−)-beta-pineen, L-asparagine, L-homoserine, L-serine, L-threonine, L-prdine, L-tyrosine, L-leucine, dopamine, taurocholic acid, tryptamine, tauroursodeoxychdic acid, glycoursodeoxycholic acid, ursodeoxycholic acid, cholic acid, nonanal, 3-methyl-2-butenal, DL-glyceraldehyde, allantoin, nicotinic acid, N-acetylglucosamine, spermidine, (dimethylamino)acetonitrile, glycoursodeoxycholic acid, tauroursodeoxycholic acid, cortisol, and heptanal.

36. The method of claim 34 or claim 35, wherein said one or more metabolite(s) are quantified by using antibodies which specifically bind to said metabolite.

37. The method of claim 36, wherein said antibodies are part of an Enzyme-Linked Immuno Sorbent Assay (ELISA).

38. The method of claim 37, wherein said one or more metabolite(s) are quantified by using mass spec or HPLC.
